(19) United States
(12) Patent Application Publication
(10) Pub. No.: US 2025/0311920 A1
(43) Pub. Date: Oct. 9, 2025

(54) NAVIGATION DEVICE AND METHODS OF USE THEREOF

(71) Applicant: Axiom Labs, Inc., San Francisco, CA (US)

(72) Inventors: Mason Alexander Sanfilippo, San Francisco, CA (US); Nathan Paul Dalva-Baird, Sacramento, CA (US); Kevin William Zhu, San Francisco, CA (US); Ian Coll McEachern, San Francisco, CA (US); Michael J. Brown, Superior, CO (US)

(21) Appl. No.: 19/172,363

(22) Filed: Apr. 7, 2025

Related U.S. Application Data
(60) Provisional application No. 63/575,583, filed on Apr. 5, 2024.

(51) Int. Cl. A61B 1/233; A61B 1/00

(52) U.S. Cl. CPC A61B 1/233; A61B 1/00078; A61B 1/00087; A61B 1/00103; A61B 1/00148; A61B 1/00154

(57) ABSTRACT

Aspects of the present disclosure include a navigation device for insertion with an insertion device into the nasal cavity and methods of use thereof. Aspects of the present disclosure includes a kit for using the navigation device. Aspects of the present disclosure include a system comprising a navigation device.

### 1. CROSS-REFERENCE TO RELATED APPLICATIONS

This application claims the benefit of U.S. Provisional Application No. 63/575,583, filed Apr. 5, 2024, which is hereby incorporated by reference in its entirety.

### 2. BACKGROUND

The cribriform plate is a thin segment of the ethmoid bone located at the anterior base of the skull, directly beneath the brain. It forms the superior boundary (ceiling) of the olfactory cleft, the anatomical space involved in the sense of smell. This bone structure contains multiple small openings (foramina) through which nerve fibers (axons) from olfactory sensory neurons extend upward into the brain to facilitate the sense of smell. The cribriform plate is susceptible to surgery-caused fracture during routine upper sinus surgery, especially along the lateral lamella.

Olfactory sensory neurons (OSNs, or ONs) are definitionally located in close proximity to the cribriform plate. Sampling or collecting tissue specimens near the cribriform plate carries inherent risk of structural damage if performed without specialized tools or adequate clinical expertise. Specifically, standard nasal swabs or improper sampling techniques have been documented to inadvertently enter the olfactory cleft and result in fractures to the cribriform plate at the skull base. Such fractures have led to cerebrospinal fluid (CSF) leaks, a serious medical complication, in several reported clinical cases. In common practice, collections of olfactory epithelial (OE) tissue happen under endoscopic visualization by a specialist practitioner. Endoscopic visualization is typically required for OE collection because stiff collection tools are risky to the cribriform plate, and soft tools cannot be navigated to the olfactory cleft without visualization. There are no specialized OE collection devices in common practice. Use and knowledge of collection of this tissue is significantly limited by the state of the art and only exists in select specialized academic studies.

Olfactory epithelium and ON collection is an important technology for neuroscience and neurological health. ONs are a class of neurons which originate on the nasal side of the skull base, but which make a synapse with the brain. It is possible to sample them from the nasal side without causing lasting harm. They are regenerative.

ONs, as a neuronal population, are shown to have diagnostic potential for neurodegenerative and neuropsychiatric disease, and they may serve as a disease model for mechanistic and translational understanding of these diseases. Collection of these cells by non-specialists increases the scalability of dataset generation for biomarker and mechanistic discovery and improves the accessibility of future diagnostic tools.

There is a need for devices and methods to aid in the navigation of an insertion device to the olfactory region of the nasal cavity. For example, there is a need for navigation of a device for collection of olfactory cells and tissues in a clinical care setting (typically a doctor's office, ambulatory clinic, hospital, lab, home, or other location where subjects are undergoing collection of OE/ONs).

### 3. SUMMARY

Aspects of the present disclosure include a navigation device for insertion of an insertion device into the nasal cavity and methods of use thereof.

An aspect of the present disclosure includes a navigation device comprising: a body comprising: a conduit comprising a proximal end, a distal end, and a lumen extending therethrough, wherein the proximal end of the conduit comprises a tip configured to be inserted into a nasal opening of a subject; a handle adjoining to the distal end of the conduit and configured to: insert the proximal end of the conduit into the olfactory cleft of the subject.

In some embodiments, the proximal end of the conduit is inserted until a depth limiter restricts further movement of the conduit through the nasal cavity of the subject. In some embodiments, the depth limiter positioned along a portion of the outer length of the conduit configured to restrict movement of the conduit within the nasal cavity when the depth limiter is in contact with the nasal apex, tip of the nose, or adjacent anatomical structures of the subject such as: major alar cartilage, nasal vestibule floor, lower lateral cartilage, septal cartilage. In some embodiments, a depth limiter positioned along a portion of the outer length of the conduit restricts further movement of the conduit through the nasal cavity of the subject when the depth limiter is in contact with the nasal tip, apex, or major alar cartilage of the subject.

In some embodiments, the device external conduit surface comprises a low surface energy material to reduce friction during device insertion.

In some embodiments, the tip is a deflection tip. In some embodiments, the navigation device is made from one or more materials. In some embodiments, the one or more materials is configured to provide one or more regions of stiffness along the length or cross-section of the conduit.

In some embodiments, the one or more materials is selected from: Polyetheretherketone (PEEK), Polycarbonate (PC), Polyetherimide (PEI, Ultem), Polysulfone (PSU), Polyphenylsulfone (PPSU), Polyamide (Nylon, PA), Acrylic (PMMA), Polyethylene Terephthalate (PET), Rigid Polyvinyl Chloride (Rigid PVC), Acrylonitrile Butadiene Styrene (ABS). In some embodiments, the one or more materials is selected from: Ethylene-vinyl acetate (EVA), Polyolefin (PO), Polyether block amide (PEBA, Pebax), Thermoplastic elastomers (TPE), Thermoplastic polyurethane (TPU), Silicone elastomer (Silicone), and Flexible polyvinyl chloride (Flexible PVC).

In some embodiments, one or more materials is configured to prevent the navigation device from exerting an amount of pressure on the nasal cavity or cribriform plate of the subject.

In some embodiments, the body comprises a proximal opening at the proximal end of the conduit. In some embodiments, the distal opening is at the distal end of the handle. In some embodiments, the conduit comprises a first curved region near the proximal end of the conduit, wherein the first curved region comprises a radius of curvature ranging from 20 mm to 175 mm. In some embodiments, the conduit comprises a first curved region at the proximal end of the conduit, wherein the first curved region comprises a radius of curvature ranging from 20 mm to 75 mm. In some embodiments, the conduit comprises a first curved region at the proximal end of the conduit, wherein the first curved region comprises a radius of curvature ranging from 40 mm to 60 mm.

In some embodiments, the conduit is configured for insertion into the olfactory cleft of the subject, wherein the first curved region of the conduit facilitates anatomical alignment to the olfactory cleft during placement. In some embodiments, the proximal end of the conduit comprises a second curved region near the deflection tip.

In some embodiments, the second curved region comprises a radius of curvature ranging from 5 mm to 100 mm. In some embodiments, the second curved region comprises a radius of curvature ranging from 50 mm to 100 mm. In some embodiments, the second curved region comprises a radius of curvature ranging from 70 mm to 90 mm.

In some embodiments, the proximal end of the conduit comprises a third curved region comprising a radius of curvature ranging from 20 mm to 150 mm. In some embodiments, the third curved region comprises a radius of curvature 70 mm to 150 mm. In some embodiments, the third curved region comprises a radius of curvature of 110 mm to 130 mm.

In some embodiments the length of the distal region of the conduit, for example the sum of lengths L1 and L2 or the sum of lengths L4 and L5 in FIG. 1B, ranges from 0-50 mm. In some embodiments the length of the distal region of the conduit, for example the sum of lengths L1 and L2 or the sum of lengths L4 and L5 in FIG. 1B, ranges from 0-40 mm. In some embodiments the length of the distal region of the conduit, for example the sum of lengths L1 and L2 or the sum of lengths L4 and L5 in FIG. 1B, ranges from 0-20 mm.

In some embodiments, the deflection tip comprises a flared tip. In some embodiments, the deflection tip comprises a relatively straight tip. In some embodiments, the deflection tip is a relatively more flexible region compared to the rest of the conduit.

In some embodiments, the conduit comprises a channel extending through the lumen. In some embodiments, the channel comprises a sample retention tip. In some embodiments, the inner diameter of the proximal opening of the conduit ranges from 0.25 mm to 5 mm. In some embodiments, the outer diameter of the proximal opening of the conduit ranges from 0.25 mm to 6 mm.

In some embodiments, the conduit is inserted within the nasal cavity until an insertion depth is reached. In some embodiments, the insertion depth is determined by an external measurement of the subject's nasal anatomy from the external nasion or glabella to the nasal apex or adjacent anatomical structures. In some embodiments, the insertion depth is determined by a calculation based on the external nasal length of the subject's nose. In some embodiments, the insertion depth ranges from 40 mm to 90 mm. In some embodiments, the depth limiter is positioned along the outer length of the conduit. In some embodiments, the depth limiter is positioned at a location along the outer length of the conduit such that, when the conduit is advanced into the nasal cavity of the subject, the depth limiter restricts the insertion depth of the conduit ranging from 40 mm to 90 mm.

In some embodiments, the depth limiter is configured to set the insertion depth range of the conduit to 40 mm to 90 mm when secured in place. In some embodiments, the depth limiter is slidable when in an unlocked position and securable when in a locked position at a selected position on the outer region of the conduit to limit movement of the conduit until the insertion depth of the conduit is reached. In some embodiments, the depth limiter comprises a locked configuration and an unlocked configuration. In some embodiments, the depth limiter comprises a locking mechanism configured to secure the depth limiter in place on the conduit once adjusted, thereby preventing movement or loosening of the depth limiter. In some embodiments, the depth limiter comprises a stepped locking mechanism, such that the depth limiter can be placed in a particular number of preset positions, ranging from 3 to 15 different positions. In some embodiments, the depth limiter is inherent in the design of the device and unmovable, and there are different devices representing different fixed lengths.

In some embodiments, the distal end of the conduit is downstream of the depth limiter. In some embodiments, the proximal end of the conduit is upstream of the depth limiter, wherein the depth limiter separates the proximal region and the distal region of the conduit. In some embodiments, the handle is configured to be rotated between 5-120 degrees along its longitudinal axis.

In some embodiments, the handle is configured to rotate about 90 degrees along its longitudinal axis. In some embodiments, the handle is configured to rotate in a counterclockwise direction along its longitudinal axis. In some embodiments, the handle is configured to rotate in a clockwise direction along its longitudinal axis. In some embodiments, the lumen and channel of the conduit extends through the handle, wherein the handle adjoins the conduit at the distal end of the conduit, and wherein the handle comprises an opening at the distal end of the handle.

In some embodiments, the navigation device further comprises an insertion device, wherein the insertion device is configured to move through the lumen of the conduit and into the olfactory region of the nasal cavity of the subject. In some embodiments, the insertion device is a collection tool configured to collect nasal cells and/or olfactory tissue. In some embodiments, the insertion device is a delivery tool configured to deliver a therapeutic agent to the olfactory region of the nasal cavity. In some embodiments, the insertion device is a surgical tool. In some embodiments, the insertion device is an imaging tool.

In some embodiments, the distal end of the conduit has an outer diameter ranging from 0.25 mm to 6 mm. In some embodiments, the distal end of the conduit has an inner diameter ranging from 0.25 mm to 5 mm.

In some embodiments, the collection tool comprises: a collection tip at the proximal end of the collection tool configured to collect the sample comprising nasal cells and/or olfactory tissue; a shaft extending from the collection tip; a handle attached to a distal end of the shaft.

In some embodiments, the collection tip comprises a swab or brush at the proximal end of the collection tool. In some embodiments, the collection tip comprises bristles at the proximal end of the collection tool. In some embodiments, the collection tip comprises kerfs at the proximal end of the collection tool. In some embodiments, the collection tip comprises a debrider at the proximal end of the collection tool.

In some embodiments, the collection tip is configured to remove, detach, and/or scrape the olfactory epithelium and/or nasal cells. In some embodiments, the nasal cells are olfactory cells. In some embodiments, the olfactory cells include: olfactory sensory neurons, olfactory supporting cells, olfactory ensheathing cells (OECs), mesenchymal stem cells, epithelial cells, and basal (stem) cells. In some embodiments, the olfactory region is the olfactory cleft of the nasal cavity.

In some embodiments, the olfactory region comprises the olfactory mucosa. In some embodiments, the collection tool is within the lumen and/or channel of the conduit. In some embodiments, the collection tool is fully extended through the lumen and channel when the handle of the collection tool is adjacent to the distal end of the handle of the conduit.

An aspect of the present disclosure includes a method of collecting a sample from the nasal cavity of a subject, the method comprising: (1) inserting a proximal end of a conduit of a navigation device of the present disclosure into the nasal opening of the subject, wherein one side of the conduit is adjacent to the septum of the nasal cavity; (2) once the conduit is in the olfactory cleft of the nasal cavity, inserting the insertion device comprising a collection tool through the lumen of the conduit and into the olfactory region of the nasal cavity of the subject; and (3) collecting the sample by rotating the collection tool.

In some embodiments, the collection tool is rotated between 0.5 to 20 complete rotations. In some embodiments, the method further comprises, before step (2), rotating the conduit with the handle between 5-90 degrees along its longitudinal axis, wherein the proximal end of the conduit is inserted to and past the nasion of the nasal cavity of the subject until the depth limiter restricts further movement of the conduit through the nasal cavity. In some embodiments, the collection tool is fully extended through the lumen and channel when the handle of the collection tool is adjacent to the distal end of the handle of the conduit.

In some embodiments, the collection tool comprises a handle at the distal end of the collection tool. In some embodiments, the collection tool is fully extended through the lumen and channel when the handle of the collection tool is adjacent to the distal end of the handle of the conduit. In some embodiments, the method comprises removing the navigation device from the nasal cavity of the subject.

In some embodiments, removing the navigation device comprises retracting the collection tool distally away from the olfactory region of the subject's nasal cavity and fully within the lumen and channel of the conduit; and removing the conduit of the navigation device out of the nasal cavity of the subject. In some embodiments, removing the conduit of the navigation device out of the nasal cavity of the subject comprises rotating the conduit. In some embodiments, the method comprises placing the collection tool in a buffer.

In some embodiments, the sample comprises olfactory tissue. In some embodiments, the sample comprises nasal cells. In some embodiments, the sample comprises sensory neuronal cells. In some embodiments, the nasal cells are olfactory cells.

In some embodiments, the olfactory cells are selected from: olfactory sensory neurons, olfactory supporting cells, olfactory ensheathing cells (OECs), olfactory epithelial cells, mesenchymal stem cells, and basal (stem) cells. In some embodiments, the olfactory region is the olfactory cleft of the nasal cavity. In some embodiments, the olfactory region comprises olfactory filaments, the olfactory epithelium, and the lamina propria of the olfactory mucosa. In some embodiments, the method further comprises analyzing the sample. In some embodiments, the analyzing the sample comprises determining the suitability of the olfactory cells within the sample for downstream applications.

In some embodiments, the method comprises, before (1) inserting a proximal end of a conduit of a navigation device of the present disclosure into the nasal opening of the subject, measuring the length from the external nasion or adjacent anatomy to the nasal apex or adjacent anatomy of the subject to determine the insertion depth of the navigation device. In some embodiments, the insertion depth is the length at which the depth limiter is positioned on the outer position of the conduit.

An aspect of the present disclosure includes a method of navigating an insertion device through a nasal cavity of a subject, the method comprising: (1) inserting a proximal end of a conduit of a navigation device of the present disclosure into the nasal opening of the subject, wherein one side of the conduit is adjacent to the septum of the nasal cavity; (2) once the conduit is in the nasion of the nasal cavity, inserting the insertion device through the lumen of the conduit and into the olfactory region of the nasal cavity of the subject; and (3) performing a function intended by the insertion device.

In some embodiments, the insertion device is a collection tool configured to collect the sample by rotating the collection tool between 0.5 to 20 complete rotations.

In some embodiments, the insertion device is a delivery tool configured to deliver a therapeutic agent to the olfactory region of the nasal cavity. In some embodiments, the insertion device is an imaging tool. In some embodiments, the insertion device is a surgical tool.

An aspect of the present disclosure includes a system for navigating a navigation device into the nasal cavity of a subject, the system comprising: a navigation device of the present disclosure; an insertion device configured to be placed through the lumen of the conduit and into the olfactory region of the nasal cavity of the subject to perform its intended function; one or more additional components configured to aid the navigation device to be inserted into the nasal cavity of the subject.

In some embodiments, one or more additional components is a nasal valve dilator.

In some embodiments, one or more additional components is a self-adhesive guide device.

An aspect of the present disclosure includes a kit for navigating a navigation device into the olfactory cleft of a subject, the kit comprising: a navigation device of the present disclosure.

In some embodiments, the kit further comprises an insertion device configured to be placed through the lumen of the conduit and into the olfactory cleft of the nasal cavity of the subject to perform its intended function.

In some embodiments, the kit comprises one or more additional components configured to aid the navigation device to be inserted into the nasal cavity of the subject.

In some embodiments, one additional component is a nasal valve dilator.

In some embodiments, one additional component is a self-adhesive guide device.

### 4. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A provides different views of the navigation device according to one embodiment of the present disclosure. FIG. 1B provides a front view of the device and a side view of the device according to one embodiment of the present disclosure.

FIG. 2 provides a front view, side views, and a back view of the navigation device according to one embodiment of the navigation device.

FIG. 3A illustrates different features of the proximal end of the conduit and the conduit tip according to several embodiments of the navigation device. FIG. 3B provides a feature of the deflection tip that is a straight feature vs a curved feature illustrated in FIG. 3A.

FIG. 3C provides a non-limiting example of an actuation method of the navigation device according to one embodiment of the present disclosure.

FIG. 4 illustrates different features of the depth limiter and the collection tool tip, such as a debrider, brush, swab, or other biopsy mechanism.

FIG. 5 shows one embodiment of the handle of the conduit as a barrel containing a captive spring assembly used to limit the force and depth of insertion of the conduit.

FIG. 6 illustrates different features of the navigation device according to one embodiment of the navigation device.

FIGS. 7A-7B provide photographs of one embodiment of the tip of the navigation device. FIG. 7A shows the navigation device in a retracted position (FIG. 7A), where the collection tip of the collection tool is protected with a cover (e.g., safety tip when the conduit is advancing to the olfactory cleft of the nasal cavity prior to collecting the sample). FIG. 7B shows the navigation device in an extended position. The extended position can include deploying or advancing the sample collection tip of the sample collection tool into the olfactory region to collect the sample.

FIGS. 8A-8B provide photographs of one embodiment of the navigation device. FIG. 8A shows the navigation device in an extended configuration. The extended position can include deploying or advancing the sample collection tip of the sample collection tool into the olfactory cleft to collect the sample. FIG. 8B is a close up of the collection tip within extending out of the lumen of the conduit of FIG. 8A. FIG. 8C shows the navigation device in a retracted position (FIG. 8C), where the collection tip of the collection tool is within the lumen or channel of the conduit of the navigation device.

FIG. 9 provides an illustration of a guiding device that can guide the navigation device into the nasal cavity according to one embodiment of the present disclosure. Alternatively, the guiding device can be the conduit of the navigation device according to one embodiment of the present disclosure.

FIGS. 10A-10C show anatomical landmarks with complex internal nasal geometry used to determine the insertion depth(s) of the conduit of the navigation device across different nasal cavities. In order for the conduit (e.g., sheath) to be inserted to the olfactory cleft through surrounding anatomical features, an external measurement is performed. FIG. 10A shows the red measurement (external nasion to nasal apex or surrounding anatomical structures) is an external measurement of the subject's nasal anatomy (EN) used to determine the insertion depth of the conduit. A portion of the path shown taken by the device shown in yellow can be described as the distance between the nasal apex or surrounding anatomical structures to the internal nasion, moving along the dorsal surface of the nasal cavity. The internal nasion is defined as the interior anatomical point where the nasal bones meet the frontal bone, posterior to the exterior nasion. The measurement from the nasal apex to the internal nasion is subject-specific. Due to the invasive nature of blind internal measurement proximal to sensitive internal anatomy of the nasal cavity, direct measurement of this section is not feasible. To accurately estimate this segment, an external linear measurement is manually taken from the external nasion to the nasal apex. This external measurement is then used as a scaling factor (also used interchangeably herein as “conversion factor”) to extrapolate the segment from the internal nasion to the nasal apex. The conduit's total insertion length is then calculated by summing literature-derived lengths with the scaled subject-specific length. This method allows precise, subject-adapted device placement without invasive measurements or procedures. FIG. 10B shows additional anatomical features of the nasal cavity. FIG. 10C provides anatomical regions of the nasal cavity where the navigation device avoids based on the design and geometry of the navigation device.

FIG. 11 provides an illustration of non-limiting exemplary steps using the navigation device according to one embodiment of the methods provided herein. Before the conduit is inserted into the nasal cavity, the external nasion to the nasal apex of the subject is measured to determine the insertion depth. The depth limiter is then adjusted and positioned on the outer length of the conduit at an insertion depth depending on the previous measurement. Step 1: The device is aligned coaxial with the nasal cavity and with the hand label “1” normal to the septal plane. The proximal end of the conduit is then inserted or advanced axially into the nasal cavity and toward the nasion of the subject until the user inserting the conduit feels a slight resistance (e.g., resistance of the conduit in contact with a wall within the nasal cavity) (steps 1-2). Once a slight resistance is felt by the user, the handle of the conduit is twisted or rotated from “1” until the handle label “2” is normal to the septal plane (e.g., about 90 degrees) (step 3). Once the indicatory channel is normal to the septal plane and the number “2” on the handle is visible, the conduit is then further advanced or inserted until the depth limiter is in contact with the nasal apex of the subject (step 4), signifying full insertion depth into the olfactory cleft. The insertion device is then extended or inserted within the lumen of the conduit until the handle of the insertion tool is adjacent or touching the distal end of the handle of the conduit (Step 5; collection tool is advanced until fully extended) such that the insertion tip reaches the olfactory region of the subject's nasal cavity. In this non-limiting example, the insertion device is a collection tool. The collection tool is rotated or twisted a number of times to collect the sample (Step 6). The navigation device is then removed or retracted from the nasal cavity to the starting position (step 7). The device is then fully retracted with axial rotation allowed (Step 8).

FIG. 12 provides an illustration of non-limiting exemplary steps using the navigation device according to one embodiment of the methods provided herein. Before the conduit is inserted into the nasal cavity, the external nasion to the nasal apex of the subject is measured to determine the insertion depth. The depth limiter is then adjusted and positioned on the outer length of the conduit at an insertion depth depending on the previous measurement. Step 1: The device is aligned coaxially with the nasal cavity. The proximal end of the conduit is then inserted or advanced axially into the nasal cavity until the depth limiter is in contact with the nasal apex of the subject (step 2), signifying full insertion depth into the olfactory cleft. The insertion device is then extended or inserted within the lumen of the conduit until the handle of the insertion device is adjacent or touching the distal end of the handle of the conduit (Step 3; collection tool is advanced until fully extended) such that the insertion tip is exposed to the olfactory cleft. In this non-limiting example, the insertion device is a collection tool. The collection tool is rotated or twisted a number of times to collect the sample, then retracted into the conduit to shroud the sample from contamination or unintended collection (Step 4). The navigation device is then removed or retracted from the nasal cavity to starting position (step 5). The device is then fully retracted with axial rotation allowed (Step 6).

FIG. 13 provides a non-limiting example of the thermoforming process used to manufacture the conduit. After extrusion into a hollow cylinder, the tube is inserted into a mold and heated to re-set the shape into the desired spline.

FIG. 14 provides an exemplary compressive load experiment of the deflection tip of the navigation device, where the device is compressed 15 mm until the deflectable tip collapses on the plate.

FIG. 15 provides a non-limiting example of an accessory component used to guide the navigation device into the nasal cavity. As shown in FIG. 15, this accessory comprises a tube with one or more lumens (1), a patch of self-adhesive tape (2), and a guide rod (3) with a lumen at a preset angle (4). The adhesive (2) is adhered to the subject's nasal ridge. The guide rod (3) is inserted into (2) until resting against the nasal apex. The insertion device, such as a biopsy device is inserted through (4) and kept at a set angle relative to the nasal bridge as a result.

FIGS. 16A-16C show different self-adhesive devices that can be used in accordance with the navigation device of the present disclosure. The accessory component as shown in FIG. 16A includes a flexible beam (1), a self-adhesive patch (2), and a guide sheath (3) containing a lumen oriented at a preset angle (4) to accommodate the insertion device such as a biopsy device. The adhesive patch (2) is applied to the subject's nasal ridge. The guide sheath (3) is advanced along the flexible beam (1) until it rests against the nasal apex. The insertion device such as a biopsy device is then inserted through the angled lumen (4), ensuring it remains at a consistent, predefined angle relative to the nasal bridge. The accessory component as shown in FIGS. 16B-16C comprises two arms containing undercut circular channels (1), oriented at a predetermined angle, and a self-adhesive patch (2). Each arm (1) is inserted into a nostril until the distal end (3) contacts the nasal apex, at which point the adhesive patch (2) is secured to the subject's nasal ridge. An insertion device such as a biopsy device is then inserted through one or both angled channels (1), maintaining a consistent, predefined angle relative to the nasal bridge throughout the procedure. FIG. 16B, (A) shows the accessory advanced until in contact with the nasal apex, while (B) and (C) show the biopsy device advanced at a predefined angle relative to the nasal bridge.

### 5. DETAILED DESCRIPTION

### 5.1. Navigation Device

### 5.1.1. Body

An aspect of the present disclosure includes a navigation device for collecting a sample within one or more particular regions of the nasal cavity, such as the olfactory cleft of the nasal cavity.

In some aspects of the present disclosure, the navigation device includes a body comprising: a conduit comprising a proximal end, a distal end, and a lumen extending therethrough; and a handle adjoining or connecting to the distal end of the conduit. In some embodiments, the conduit comprises a channel extending through the lumen of the conduit.

In some embodiments, the body comprises a proximal opening at the proximal end of the conduit and a distal opening at the distal end of the handle. In some embodiments, the body comprises a length ranging from 50 to 250 mm. In some embodiments, the body comprises a length ranging from 60 to 120 mm.

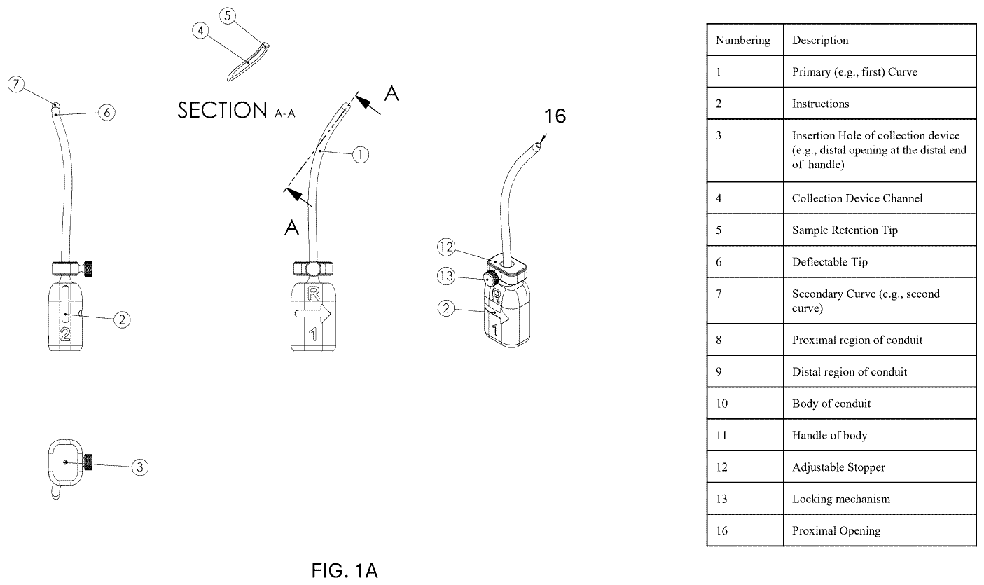

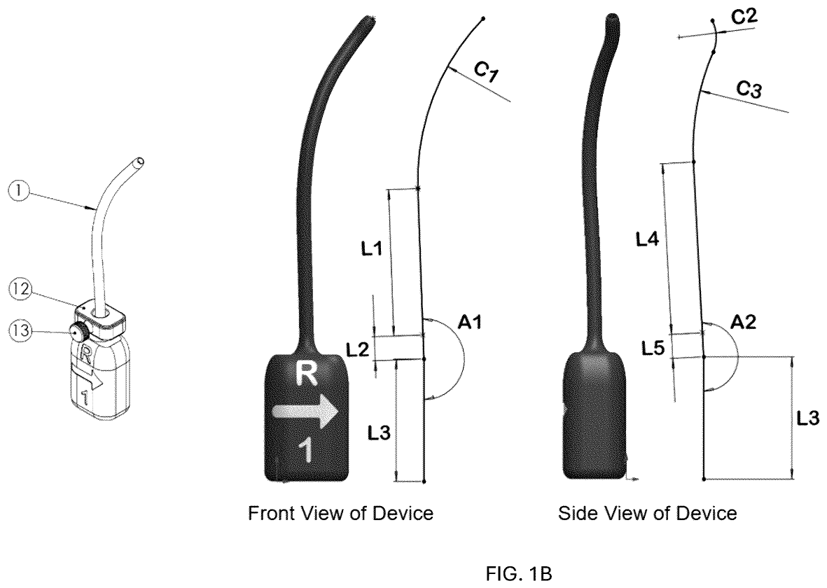

As shown in FIG. 1, the body (10) of the navigation device is provided, where the body is from the tip (6) of conduit (20) at the proximal end of the conduit to the distal end of the handle (11).

In some embodiments, the navigation device is coated with a biocompatible sterile lubricant for use.

### 5.1.1.1 Conduit

In some embodiments, the conduit of the navigation device is considered as a guide for the insertion device positioned within the lumen of the conduit, such that the insertion device is properly and safely inserted to the olfactory cleft of the subject's nasal cavity. In some embodiments, the conduit of the navigation device is considered as a sheath that protects the soft tissue and bony structures within the nasal cavity, e.g., mucosa and cribriform plate, and guides the device from inadvertently sampling the wrong area.

In some embodiments, the proximal end of the conduit is configured to be inserted into the nasal opening of the subject. In some embodiments, the conduit includes a lumen and/or channel for insertion of the insertion device within the conduit. In some embodiments, the proximal end of the channel comprises a sample retention tip. In some embodiments, the conduit is a soft material that may be customized into different lengths, diameters, cross-sectional shapes, and topologies for different nostril and nasal anatomies. Non-limiting examples of materials for which the conduit can be made from include, but are not limited to: metals, stiff plastics, flexible plastics, and polymer-based or electrically conductive coatings. In some embodiments, the metal is selected from: brass, copper, aluminum, stainless steel, titanium, and nickel-titanium alloys

In some embodiments, the conduit is made from a material selected from one or more of: Polyetheretherketone (PEEK), Polycarbonate (PC), Polyetherimide (PEI, Ultem), Polysulfone (PSU), Polyphenylsulfone (PPSU), Polyamide (Nylon, PA), Acrylic (PMMA), Polyethylene Terephthalate (PET), Rigid Polyvinyl Chloride (Rigid PVC), and Acrylonitrile Butadiene Styrene (ABS).

In some embodiments, the conduit is made from a material selected from one or more of: Ethylene-vinyl acetate (EVA), Polyolefin (PO), Polyether block amide (PEBA, Pebax), Thermoplastic elastomers (TPE), Thermoplastic polyurethane (TPU), Silicone elastomer (Silicone), and Flexible polyvinyl chloride (Flexible PVC).

In some embodiments, the conduit is coated with a material selected from one or more of: PTFE (Teflon®), Hydrophilic (PVP, PEG-based), Silicone-based material, FEP, and Parylene.

In some embodiments, conduit can be made from steed braid reinforcement as a composite with stiff or flexible plastics, in combination with lubricity coatings. In some embodiments, low-friction plastic coatings (e.g., PTFE, FEP) are used on the inside surface of the conduit to allow low-friction transit of the insertion device inside of a smaller diameter lumen than otherwise is possible. Minimizing the internal diameter of the lumen allows for reduction of the overall cross section of the conduit. Reduced overall cross section of the conduit minimizes user pain and difficulty of insertion past narrow portions of the nasal cavity such as the nasal valve and the septal swell in order to reach the olfactory cleft.

In some embodiments, low-friction plastic coatings (e.g., PTFE, FEP) are used on the outer surface of the shaft to allow smoother insertion of the device into the nasal cavity and less trauma to the mucosal surface from pulling and friction. Reduced friction of the conduit minimizes user pain and difficulty of insertion past narrow portions of the nasal cavity such as the nasal valve and the septal swell in order to reach the olfactory cleft.

In some embodiments, the conduit is designed with variable stiffness and directional bending along the conduit's axial length or radial cross-section. In some embodiments, the conduit comprises multiple laminations of similar or dissimilar polymers arranged in axial or radial orientations to create localized stiffness variations. In some embodiments, the conduit exhibits axial variations in polymer crystallinity to provide mechanical stiffness gradients along its length. In some embodiments, the conduit comprises polymer blends with controlled ratios of two or more constituent materials mixed to form continuous or stepwise stiffness gradients. In some embodiments, the conduit comprises variable-density polymers with spatially controlled material density to influence local flexibility or rigidity, including via selective laser sintering. In some embodiments, the conduit includes continuous reinforcement fibers (e.g., glass, aramid, or carbon) embedded at varying orientations and densities along the tube length, enabling localized differences in stiffness. In some embodiments, the conduit contains embedded mesh or coil reinforcement structures formed from stainless steel, nitinol, or other biocompatible metals or alloys.

In some embodiments, the conduit is made of one or more materials described herein. The one or more materials from which the conduit is made is configured to prevent the navigation device from exceeding an amount of pressure on the nasal cavity or cribriform plate of the subject. In some embodiments, the one or more materials from which the conduit is made may be configured to prevent the navigation device from exceeding a maximum value between 750-4500 kPa. In some embodiments, the one or more materials from which the conduit is made may be configured to prevent the navigation device from exceeding a maximum value between 750-2000 kPa. In some embodiments, the amount of pressure is 4250 kPa or less, 4000 kPa or less, 3750 kPa or less, 3500 kPa or less, 3250 kPa or less, 3000 kPa or less, 2750 kPa or less, 2500 kPa or less, 2250 kPa or less, or 2000 kPa or less, or 1800 kPa or less.

The navigation device of claim 9, wherein the navigation device remains usable when a force applied to the navigation device is less than 10 newtons, less than 9 newtons, less than 8 newtons, less than 7 newtons, less than 6 newtons, less than 5 newtons, less than 4 newtons, or less than 3 newtons. In some embodiments, sections of the navigation device deform when a force applied to the navigation device is more than 20 newtons, more than 18 newtons, more than 16 newtons, more than 14 newtons, more than 12 newtons, more than 10 newtons, more than 8 newtons, more than 6 newtons, more than 4 newtons, more than 3 newtons, more than 2 newtons, or more than 1 newton. In some embodiments, sections of the navigation device begin to deform when a force applied to the navigation device ranges from 1-20 newtons. In some embodiments, sections of the navigation device begin to deform when a force applied to the navigation device ranges from 1-18 newtons. In some embodiments, sections of the navigation device begin to deform when a force applied to the navigation device ranges from 1-16 newtons. In some embodiments, sections of the navigation device begin to deform when a force applied to the navigation device ranges from 1-14 newtons. In some embodiments, sections of the navigation device begin to deform when a force applied to the navigation device ranges from 1-12 newtons. In some embodiments, sections of the navigation device begin to deform when a force applied to the navigation device ranges from 1-10 newtons. In some embodiments, sections of the navigation device begin to deform when a force applied to the navigation device ranges from 1-8 newtons. In some embodiments, sections of the navigation device begin to deform when a force applied to the navigation device ranges from 1-6 newtons. In some embodiments, sections of the navigation device begin to deform when a force applied to the navigation device ranges from 1-4 newtons. In some embodiments, sections of the navigation device begin to deform when a force applied to the navigation device ranges from 1-2 newtons.

In some embodiments, the navigation device remains usable when a force applied to the navigation device is less than 10 newtons, less than 9 newtons, less than 8 newtons, less than 7 newtons, less than 6 newtons, less than 5 newtons, less than 4 newtons, or less than 3 newtons. In some embodiments, remaining usable means the conduit does not kink, the conduit shape is still in specifications which can enter the olfactory cleft, or the insertion device can still be inserted.

In some embodiments, the conduit is comprised of two or three parallel lumens, where the central lumen is the main channel for the insertion device, and the adjacent lumen or lumens are configured to receive a guidewire, which can be tensioned to change the curvature of the conduit in particular regions. In some embodiments, one such guidewire selectively adjusts the curvature of the conduit in a predetermined region to facilitate alignment of the proximal end with the olfactory cleft

In some embodiments, the proximal end of the conduit comprises a tip. In some embodiments, the tip is a deflection tip.

As shown in FIG. 1A-1C, in some embodiments of the navigation device, the conduit (20) comprises a deflection tip (6) at the proximal end 8 of the conduit.

In some embodiments, the proximal end of the channel of the conduit (20) comprises a sample retention tip (5). In some embodiments, the sample retention tip (5) is configured to retain the sample collected from the sample retention tip once the sample collection tool is retracted back into the lumen and/or channel of the conduit. The sample retention tip prevents cells from sloughing off of the brush during retraction of the brush directly after sampling and thus maximizes the successful extraction of cells when used in combination with the insertion device. The sample retention tip may be constructed from low surface energy materials that minimize sloughing of the cells during retraction. The sample retention tip may also have mechanical features such as diametral changes or cuts to change the direction and magnitude of pressure exerted on the sample during retraction of the insertion device such as a collection tool.

In some embodiments, the body comprises a proximal opening (16) at the proximal end of the conduit and a distal opening at the distal end of the handle. An example of the distal opening of the body is provided as (3) in FIG. 1. In some embodiments, the distal opening has an inner diameter ranging from 0.25 mm to 5 mm. In some embodiments, the proximal opening has an inner diameter ranging from 0.25 mm to 2 mm. In some embodiments, the distal opening has an outer diameter ranging from 0.25 mm to 6 mm. In some embodiments, the distal opening outer diameter is defined as the handle outer diameter, with a maximum of 75 mm. In some embodiments, the proximal opening has an outer diameter ranging from 0.2 mm to 5 mm.

### 5.1.1.1.1 Proximal End of Conduit

In some embodiments, the proximal end of the conduit comprises a deflection tip. In some embodiments, the deflection tip is defined as configuring the conduit to compress or deform under varying force or pressure values applied by regions of the internal nasal anatomy, especially near the olfactory cleft. In some embodiments, the deflection tip is a more flexible region of the conduit in the last 5 mm-20 mm proximally. The deflectable tip has three primary functions: topological compliance, force limitation, and angle of the tip once the navigation device is positioned in the olfactory cleft. First, it increases subject comfort by complying with the topology of the nasal cavity as it is inserted and moved into position. Second, it increases subject safety by limiting the force value applicable to sensitive bony or soft tissue structures in the nasal cavity including the cribriform plate, turbinates, and sphenoid among other structures. Third, it allows for the internal anatomy to affect the angle of the tip once the navigation device is positioned in the olfactory cleft, including but not limited to the purpose of modifying the exit angle of a sampling brush (e.g., collection tip of collection tool) to move inferior to the cribriform plate as the brush moves posterior in the cavity. This reduces risk of applying excessive load to the cribriform plate by complete avoidance in addition to force limitation and may increase sample quality from the possible ability to extend the brush further into the cavity, collecting more cells and increasing the “sample window”.

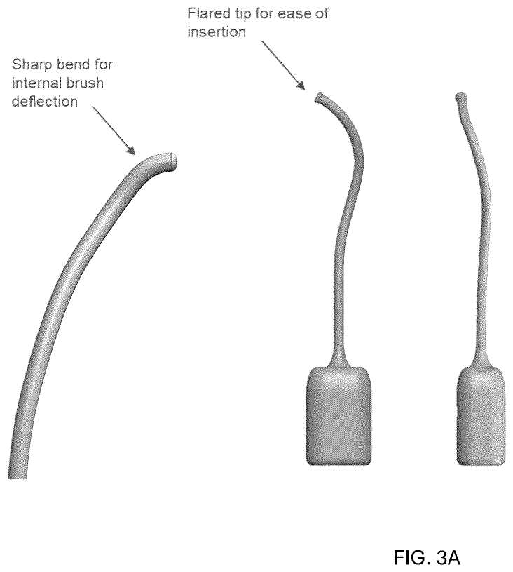

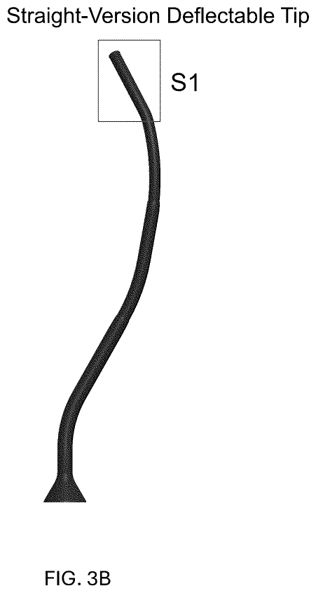

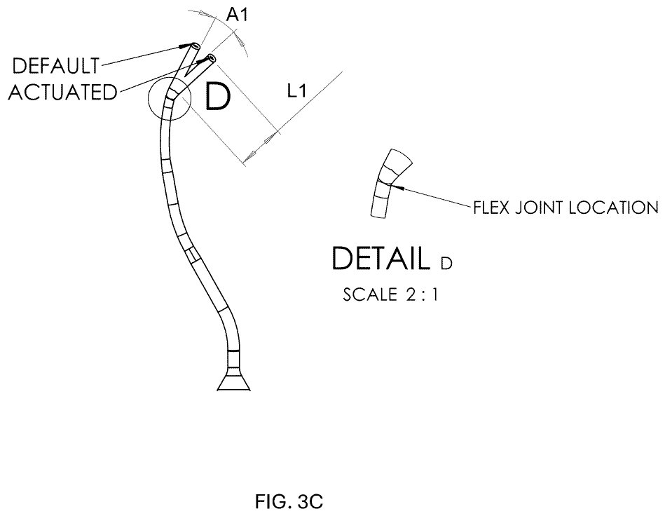

As shown in FIG. 3C, the deflection tip may be actuated via guidewire in one or more lumens of the navigation device. The actuated portion is shown in “D” of FIG. 3C. In some embodiments, “L1” of FIG. 3C is a length ranging from 0 mm to 50 mm. In some embodiments, “L1” of FIG. 3C is a length ranging from 0 mm to 20 mm. In some embodiments, “A1” of FIG. 3C comprises a degree ranging from 180 to 225 degrees.

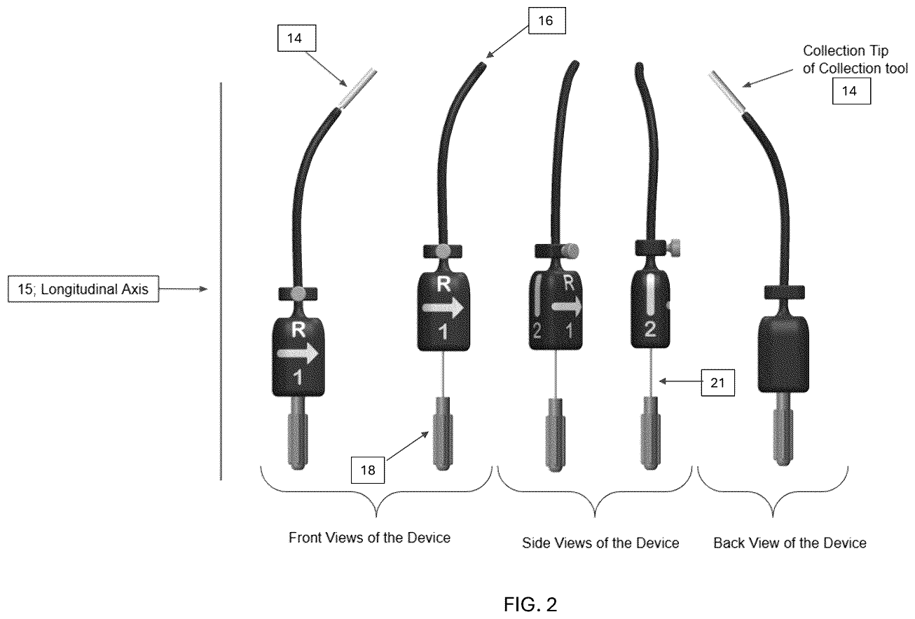

In some embodiments, the proximal end of the conduit comprises a sample retention tip (5) as shown in FIG. 1. In some embodiments, the proximal end of the conduit comprises a primary curved region, used interchangeably herein as “first curved region” or “primary curve” or “first curvature” on the conduit. In some embodiments, this first curve bends downwards or to the rear, compared to the distal portion of the conduit, as viewed in the final orientation of the device when inserted. An example of the first curved region is provided as 1 of FIG. 1. In some embodiments, the first curved region has a radius of curvature between 20 mm to 175 mm relative to a longitudinal axis (15 of FIG. 2) of the conduit. In some embodiments, the first curved region of the conduit comprises a radius of curvature ranging from 20 mm to 175 mm. In some embodiments, the first curved region of the conduit comprises a radius of curvature ranging from 40 mm to 60 mm. In some embodiments, the first curved region of the conduit comprises a radius of curvature ranging from 20 mm to 50 mm. In some embodiments, the first curved region of the conduit comprises a radius of curvature ranging from 20 mm to 80 mm. In some embodiments, the first radius of curvature is shown as “C1” of FIG. 1B. In some embodiments, the first curved region of the conduit comprises a radius of curvature of about 20 mm, about 21 mm, about 22 mm, about 23 mm, about 24 mm, about 25 mm, about 26 mm, about 27 mm, about 28 mm, about 29 mm, about 30, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm. In some embodiments, the first curved region of the conduit comprises a radius of curvature of about 20 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, about 95 mm, about 100 mm, about 105 mm, about 110 mm, about 115 mm, about 120 mm, about 125 mm, about 130 mm, about 135 mm, about 140 mm, about 145 mm, about 150 mm, about 155 mm, about 160 mm, about 165 mm, about 170 mm, or about 175 mm.

In some embodiments, the first curved region extends over a predetermined length from an initial point of curvature to a terminal point of curvature. In some embodiments, the first curved region of the conduit facilitates anatomical alignment to the olfactory cleft during placement. In some embodiments, the conduit begins straight or with less curvature in this region, and one or more guidewires in secondary lumens can be tensioned to increase the curvature to create this first curve. In some embodiments, this tensioning is activated passively during use, or actively by the user.

In some embodiments, the proximal end of the conduit comprises a first curvature extending along an arc relative to a longitudinal axis (15 of FIG. 2) of the conduit ranging from 0 degrees to 50 degrees (e.g., about 0 degrees, about 2 degrees, about 4 degrees, about 6 degrees, about 8 degrees, about 10 degrees, about 12 degrees, about 14 degrees, about 16 degrees, about 18 degrees, about 20 degrees, about 22 degrees, about 24 degrees, about 28 degrees, about 30 degrees, about 32 degrees, about 34 degrees, about 36 degrees, about 38 degrees, about 40 degrees, about 42 degrees, about 44 degrees, about 46 degrees, about 48 degrees, or about 50 degrees). In some embodiments, the proximal end of the conduit comprises a first curvature extending along an arc relative to a longitudinal axis (15 of FIG. 2) of the conduit ranging from 45 degrees to 75 degrees. In some embodiments, the proximal end of the conduit comprises a first curvature ranging from 50 degrees to 75 degrees. In some embodiments, the proximal end of the conduit comprises a first curvature ranging from 55 degrees to 75 degrees. In some embodiments, the proximal end of the conduit comprises a first curvature ranging from 60 degrees to 75 degrees. In some embodiments, the proximal end of the conduit comprises a first curvature ranging from 65 degrees to 75 degrees.

In some embodiments, the proximal end of the conduit comprises a length ranging from 20 mm to 90 mm mm. In some embodiments, the proximal end of the conduit comprises a length ranging from 40 mm to 90 mm. In some embodiments, the proximal end of the conduit comprises a length ranging from 20 mm to 50 mm. In some embodiments, the proximal end of the conduit comprises a length ranging from 20 mm to 30 mm. In some embodiments, the proximal end of the conduit is shown as “C1” of FIG. 1B. In some embodiments, the proximal end of the conduit comprises a length of about 20 mm, about 21 mm, about 22 mm, about 23 mm, about 24 mm, about 25 mm, about 26 mm, about 27 mm, about 28 mm, about 29 mm, about 30, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm. mm. In some embodiments, the proximal end of the conduit comprises a length of about 20 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, about 95 mm, about 100 mm, about 105 mm, about 110 mm, about 115 mm, about 120 mm, about 125 mm, about 130 mm, about 135 mm, about 140 mm, about 145 mm, about 150 mm, about 155 mm, about 160 mm, about 165 mm, about 170 mm, or about 175 mm. In some embodiments, the proximal end of the conduit comprises a second curved region (e.g., “second curvature” “secondary curvature”, etc.) extending along an arc relative to a longitudinal axis (15 of FIG. 2) of the conduit near the proximal or deflection tip of the conduit. In some embodiments, this second curve bends towards the lateral side (according with the nostril it is inserted into), as viewed in the final orientation of the device when inserted. In some embodiments, the proximal end of the conduit comprises a second curvature ranging from 0 degrees to 50 degrees (e.g., about 1 degrees, about 2 degrees, about 4 degrees, about 6 degrees, about 8 degrees, about 10 degrees, about 12 degrees, about 14 degrees, about 16 degrees, about 18 degrees, about 20 degrees, about 22 degrees, about 24 degrees, about 26 degrees, about 28 degrees, about 30 degrees, about 32 degrees, about 34 degrees, about 36 degrees, about 38 degrees, about 40 degrees, about 42 degrees, about 44 degrees, about 46 degrees, about 48 degrees, or about 50 degrees). In some embodiments, the second curved region has a radius of curvature ranging from 5 mm to 120 mm. In some embodiments, the second curved region has a radius of curvature ranging from 70 mm to 90 mm. In some embodiments, the second curved region has a length ranging from 5 mm to 50 mm. In some embodiments, the second curved region has a radius of curvature ranging from 5 mm to 45 mm. In some embodiments, the second curved region has a radius of curvature ranging from 5 mm to 40 mm. In some embodiments, the second curved region has a radius of curvature ranging from 5 mm to 20 mm. In some embodiments, the second curved region of the conduit comprises a radius of curvature of about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, or about 15 mm. In some embodiments, the second curved region has a length of about 20 mm, about 21 mm, about 22 mm, about 23 mm, about 24 mm, about 25 mm, about 26 mm, about 27 mm, about 28 mm, about 29 mm, about 30, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm. In some embodiments, the second curved region has a radius of curvature defined as “C2” in FIG. 1B.

In some embodiments, the conduit begins straight or with less curvature in this region, and one or more guidewires in secondary lumens can be tensioned to increase the curvature to create this second curve. In some embodiments, this tensioning is activated passively during use, or actively by the user.

In some embodiments, the proximal end of the conduit comprises a third curved region. In some embodiments, this third curve bends towards the septal side (according with the nostril it is inserted into), as viewed in the final orientation of the device when inserted. In some embodiments, the third curved region is between the first curved region and the second curved region (e.g., deflection tip), as defined as “C3” of FIG. 1B. In some embodiments, the third curved region comprises a radius of curvature ranging from 20 mm to 150 mm. In some embodiments, the third curved region comprises a radius of curvature ranging from 110 mm to 130 mm. In some embodiments, the third curved region comprises a radius of curvature ranging from 20 mm to 70 mm. In some embodiments, the third curved region comprises a radius of curvature ranging from 30 mm to 60 mm. In some embodiments, the third degree of curvature of the conduit comprises a radius of curvature of about 20 mm, about 21 mm, about 22 mm, about 23 mm, about 24 mm, about 25 mm, about 26 mm, about 27 mm, about 28 mm, about 29 mm, about 30, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm. mm. In some embodiments, the third curved region of the conduit comprises a length of about 20 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, about 95 mm, about 100 mm, about 105 mm, about 110 mm, about 115 mm, about 120 mm, about 125 mm, about 130 mm, about 135 mm, about 140 mm, about 145 mm, or about 150 mm.

In some embodiments, the conduit begins straight or with less curvature in this region, and one or more guidewires in secondary lumens can be tensioned to increase the curvature to create this third curve. In some embodiments, this tensioning is activated passively during use, or actively by the user.

In some embodiments, the deflection tip of the conduit comprises a straight region as an alternative to a curved region of the deflection tip. An example of a straight, or straight-like deflection tip is provided as FIG. 3B. In this example, the flexible portion of the proximal region of the device (e.g., “C2” and “C3” of FIG. 1B) is relatively straight instead of curved.

### 5.1.1.1.2 Distal End of Conduit

The navigation device of the present disclosure includes a distal end 9 of FIG. 1A or L1 of FIG. 1B or L1+L2 of FIG. 1B of the conduit 20.

In some embodiments, the length of the distal region of the conduit ranges from 20 mm to 70 mm. In some embodiments, the length of the distal region of the conduit ranges from 0 mm to 50 mm. In some embodiments, the length of the distal region of the conduit ranges from 0 mm to 20 mm. In some embodiments, the length of the distal region of the conduit ranges from 10 mm to 70 mm. In some embodiments, the length of the distal region of the conduit ranges from 15 mm to 50 mm. In some embodiments, the length of the distal region of the conduit ranges from 20 mm to 50 mm. In some embodiments, the distal end of the conduit comprises a length of about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm . . .

In some embodiments, the distal end comprises a length L2 as shown in FIG. 1B between the handle of the navigation device and the body of the navigation device. In some embodiments, the length L2 ranges from 0 mm to 50 mm. In some embodiments, the length L2 ranges from 0 mm to 20 mm. In some embodiments, the length L2 ranges from 1 mm to 20 mm. In some embodiments, the length L2 of the conduit comprises a length of about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm.

In some embodiments, as shown in a side view of the navigation device of FIG. 1B, the navigation device comprises a length “L4” ranging from 5 mm to 70 mm. In some embodiments, as shown in a side view of the navigation device of FIG. 1B, the navigation device comprises a length “L4” ranging from 0 mm to 50 mm. In some embodiments, as shown in a side view of the navigation device of FIG. 1B, the navigation device comprises a length “L4” ranging from 0 mm to 20 mm. In some embodiments, as shown in a side view of the navigation device of FIG. 1B, the navigation device comprises a length “L4” ranging from 5 mm to 55 mm. In some embodiments, the length L4 is about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, or about 15 mm. In some embodiments, the length L4 is about 20 mm, about 21 mm, about 22 mm, about 23 mm, about 24 mm, about 25 mm, about 26 mm, about 27 mm, about 28 mm, about 29 mm, about 30, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, or about 55 mm.

In some embodiments, as shown in a side view of the navigation device of FIG. 1B, the navigation device comprises a length “L5” ranging from 0 mm to 50 mm. In some embodiments, as shown in a side view of the navigation device of FIG. 1B, the navigation device comprises a length “L5” ranging from 0 mm to 20 mm. In some embodiments, the length L5 is about 1 mm, about 2 mm, about 3 mm, about 4 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, or about 15 mm. In some embodiments, the length L5 is about 20 mm, about 21 mm, about 22 mm, about 23 mm, about 24 mm, about 25 mm, about 26 mm, about 27 mm, about 28 mm, about 29 mm, about 30, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, or about 55 mm.

In some embodiments, the proximal end of the conduit is upstream of the depth limiter, and the distal end of the conduit is downstream of the depth limiter, wherein the depth limiter separates the proximal region and the distal region of the conduit.

As shown in FIG. 1B, in some embodiments, the distal end of the conduit comprises an angle having a degree A1 ranging from 170 to 250 degrees. As shown in FIG. 1B, in some embodiments, the distal end of the conduit comprises an angle having a degree A1 ranging from 180 to 225 degrees. As shown in FIG. 1B, in some embodiments, the distal end of the conduit comprises an angle having a degree A2 ranging from 170 to 250 degrees. As shown in FIG. 1B, in some embodiments, the distal end of the conduit comprises an angle having a degree A2 ranging from 180 to 225 degrees. In some embodiments, the distal end of the conduit comprises an angle having a degree A1 ranging from 175 to 200 degrees. In some embodiments, the distal end of the conduit comprises an angle having a degree A2 ranging from 175 to 200 degrees. In some embodiments, the position of A1 and A2 on the conduit are defined as the maximum depth of engagement for the device. A1 and A2 involve the point where the handle joins the external portion of the conduit. The purpose of both of these angles is to improve user experience, subject comfort, and sample quality. The method of these improvements involves changing the angle of the visible portion of the conduit relative to the handle (and internal portion of the conduit within the handle). These changes shift the curved portions of the device (C1, C2, C3) toward the dorsal (uppermost) surface of the nasal cavity. This shift helps avoid pressure on more constricted areas (e.g. nasal valves or septal swell) that may be sensitive and allows the user to approach the nasal cavity with a more comfortable (shallower, straighter) wrist angle. There are two angles, A1 and A2, because there are two line segments (L2 and L5) that are defined relative to the central axis of the handle and internal conduit (L3).

### 5.1.1.2 Insertion Depth

In some embodiments, the conduit of the navigation device is inserted within the nasal cavity until an insertion depth is reached.

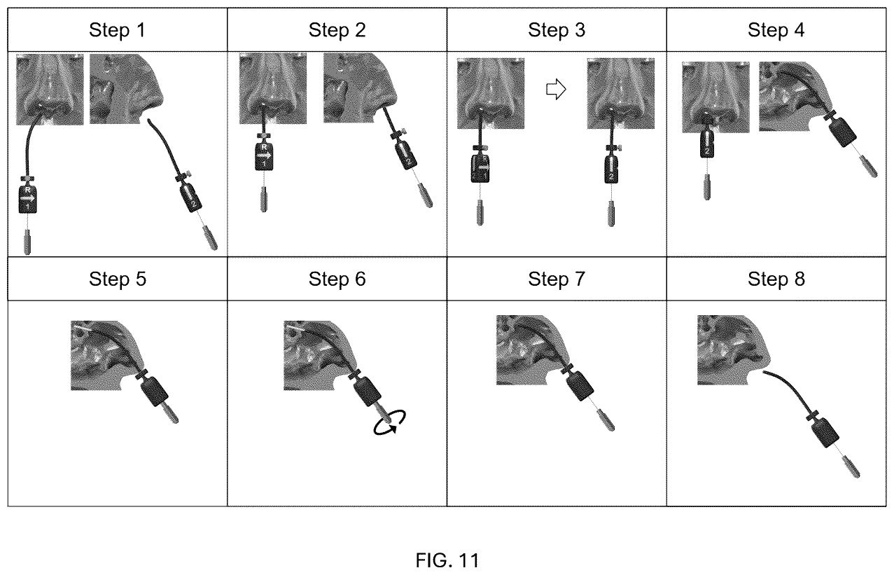

In some embodiments, the insertion depth is determined by an external nasal spline length measurement of the subject's nose. In some embodiments, the spline external nasal length measurement is a sum of the length of the external nasion to the nasal tip or nasal apex of the subject. In some embodiments, the insertion depth is the external nasal length. The device determines the total insertion length of a conduit (e.g., sheath) intended for placement within a subject's nasal cavity, specifically targeting the olfactory cleft. The calculation is the sum of three distinct anatomical segments as shown in FIG. 11: the nostril opening to the nasal apex, the nasal apex to the nasion, and the nasion to the olfactory cleft. The length from the internal nasion to the centroid of the Olfactory Epithelium is derived from existing anatomical literature. The segment from the nasal apex to the internal nasion is subject specific and not directly measurable. To accurately estimate this segment, an external measurement is manually taken from the nasion to the nasal apex and used to extrapolate internal path length based on a chart or formula. This external measurement is then used as a conversion factor to extrapolate the internal path length. The conduit's total insertion length is thus calculated by summing the literature-derived lengths with the scaled subject-specific length. This method allows precise, subject-adapted device placement without invasive measurements or procedures. The total insertion length can be calculated using the following formula:

$$TI = IN + OE + NA , where NA = EN \times SF ,$$

$$and wherein TI , IN , OE , EN and SF are defined as :$$

$$Total Insertion Length = TI ;$$

$$Olfactory epithelium ( OE ) Border to OE Centroid ;$$

$$Internal Nasion to Nasal Apex = NA = EN \times SF ;$$

$$Scaling Factor ( conversion factor ) = SF ;$$

$$Internal Nasion to OE Border = IN ; and$$

$$External Nasion to Nasal Apex = EN ( external linear measurement from provider ) .$$

In some embodiments, the length of the region containing the olfactory epithelium (OE) along the sagittal axis ranges from 2 mm to 40 mm. In some embodiments, the olfactory epithelium (OE) ranges from 4 mm to 35 mm. In some embodiments, the olfactory epithelium (OE) is at least 2 mm, at least 3 mm, at least 4 mm, at least 5 mm, at least 6 mm, at least 7 mm, at least 8 mm, at least 9 mm, at least 10 mm, at least 11 mm, at least 12 mm, at least 13 mm, at least 14 mm, at least 15 mm, at least 16 mm, at least 17 mm, at least 18 mm, at least 19 mm, at least 20 mm, at least 21 mm, at least 22 mm, at least 23 mm, at least 24 mm, at least 25 mm, at least 26 mm, at least 27 mm, at least 28 mm, at least 29 mm, at least 30 mm, at least 31 mm, at least 32 mm, at least 33 mm, at least 34 mm, at least 35 mm, at least 36 mm, at least 37 mm, at least 38 mm, at least 39 mm, or at least 40 mm.

In some embodiments, the scaling factor ranges from 0.5 to 2. In some embodiments, the scaling factor ranges from 0.6 to 2. In some embodiments, the scaling factor ranges from 0.7 to 2. In some embodiments, the scaling factor ranges from 0.8 to 2. In some embodiments, the scaling factor ranges from 0.9 to 2. In some embodiments, the scaling factor ranges from 0.5 to 1.9. In some embodiments, the scaling factor ranges from 0.5 to 1.8. In some embodiments, the scaling factor ranges from 0.5 to 1.7. In some embodiments, the scaling factor ranges from 0.5 to 1.6. In some embodiments, the scaling factor ranges from 0.5 to 1.5. In some embodiments, the scaling factor ranges from 0.5 to 1.4. In some embodiments, the scaling factor ranges from 0.5 to 1.3. In some embodiments, the scaling factor ranges from 0.5 to 1.2. In some embodiments, the scaling factor ranges from 0.5 to 1.1. In some embodiments, the scaling factor ranges from 0.5 to 1. In some embodiments, the scaling factor ranges from 0.5 to 0.9. In some embodiments, the scaling factor ranges from 0.9 to 0.99.

In some embodiments, the internal nasion to OE border ranges from 1 mm to 35 mm. In some embodiments, the internal nasion to OE border ranges from 2 mm to 35 mm. In some embodiments, the internal nasion to OE border ranges from 4 mm to 35 mm. In some embodiments, the internal nasion to OE border ranges from 6 mm to 35 mm. In some embodiments, the internal nasion to OE border is at least 8 mm, at least 10 mm, at least 12 mm, at least 14 mm, at least 16 mm, at least 18 mm, at least 20 mm, at least 22 mm, at least 24 mm, at least 26 mm, at least 28 mm, at least 30 mm, at least 32 mm, or at least 34 mm.

A non-limiting example of calculating the spline length is provided below, where the EN is measured by the provider to be 30 mm, the SF is 0.957, the IN is 12.03 mm, and the OE is 15.4 mm. Input: EN=30 mm; NA=EN×SF=28.71.

TI=IN+OE+NA=12.03+15.4+28.71=56.14 mm. Therefore, the spline length is 56.14 mm, and the depth limiter is adjusted to the equivalent position along the conduit.

In some embodiments, users are provided with a prefilled calculation chart with TI precalculated from various measured NA buckets. In some embodiments, these results correspond to distinct positions of the depth limiter. In some embodiments, these results correspond with distinct devices with different conduit lengths and insertion depths.

In some embodiments, the insertion depth ranges from 20 mm to 90 mm. In some embodiments, the insertion depth ranges from 40 mm to 90 mm. In some embodiments, the insertion depth ranges from 10 mm to 100 mm. In some embodiments, the insertion depth ranges from 30 mm to 70 mm. In some embodiments, the insertion depth ranges from 40 mm to 60 mm. In some embodiments, the insertion depth ranges from 15 mm to 80 mm. In some embodiments, the insertion depth ranges from 1 mm to 5 mm. In some embodiments, the insertion depth is about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, or about 90 mm.

### 5.1.1.3 Handle

The navigation device of the present disclosure includes a handle, where the handle adjoins or is connected to the distal end of the conduit.

In some embodiments, the handle is configured to facilitate and insert the proximal end of the conduit into the olfactory cleft of the subject until a depth limiter restricts further movement of the conduit through the nasal cavity of the subject.

In some embodiments, the handle 11 is configured to allow for rotation of the conduit about its longitudinal axis as shown in FIG. 11.

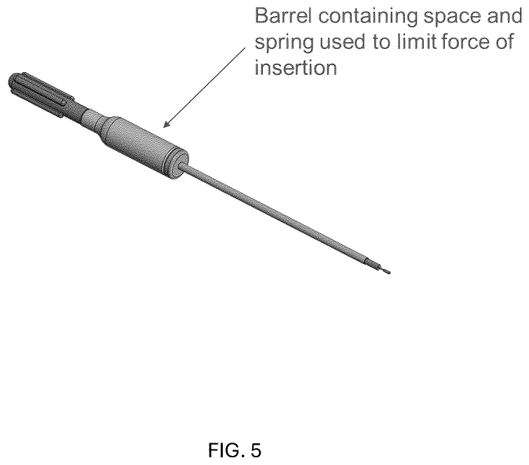

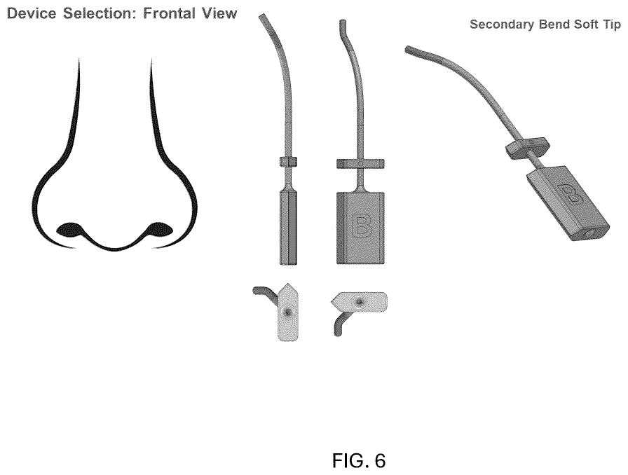

In some embodiments, the handle is a barrel-containing space comprising a spring configured to limit the force of insertion of the conduit within the nasal cavity of the subject as shown in FIG. 5.

In some embodiments, the handle is configured to rotate between 5-90 degrees along its longitudinal axis. In some embodiments, the handle is configured to rotate at least 5 degrees, at least 10 degrees, at least 15 degrees, at least 20 degrees, at least 25 degrees, at least 30 degrees, at least 35 degrees, at least 40 degrees, at least 45 degrees, at least 50 degrees, at least 55 degrees, at least 60 degrees, at least 65 degrees, at least 70 degrees, at least 75 degrees, at least 80 degrees, at least 85 degrees, or at least 90 degrees along its longitudinal axis. A non-limiting example of a navigation device with a handle that is rotated during use is provided in FIG. 11.

In some embodiments, the handle is configured to rotate in a counterclockwise direction along its longitudinal axis. In some embodiments, the handle is configured to rotate in a clockwise direction along its longitudinal axis. In some embodiments, the direction of rotation (e.g., counterclockwise or clockwise) may depend on the nostril by which the navigation device is inserted into.

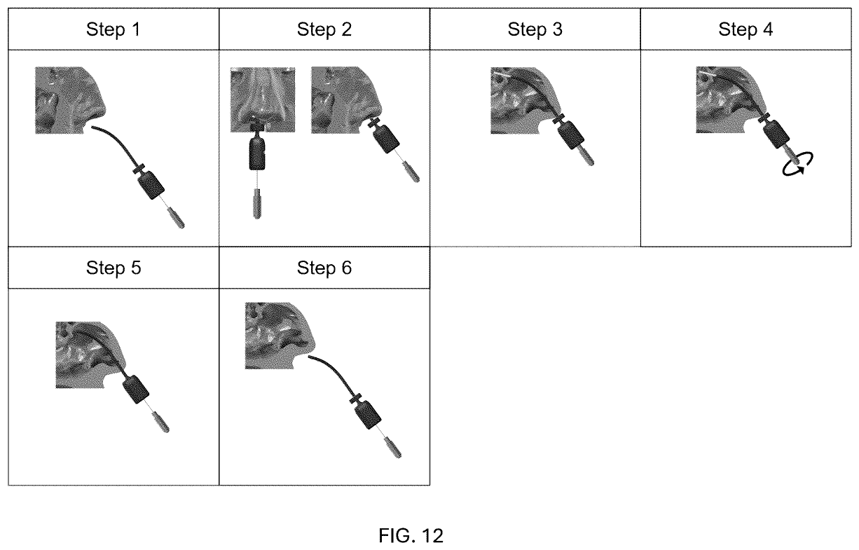

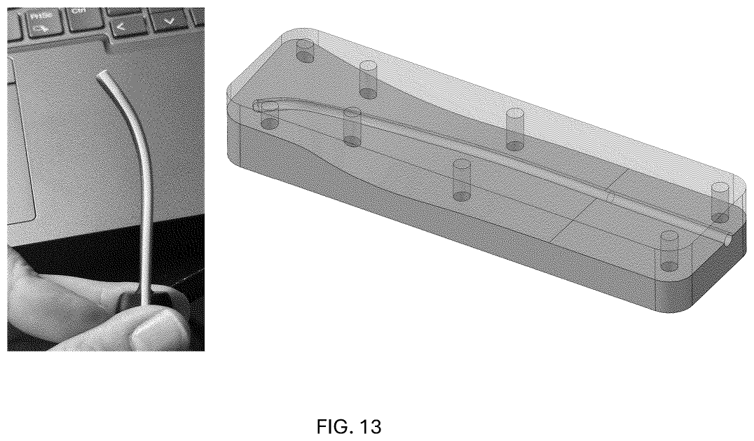

In some embodiments, the handle is not rotated axially during use. In some embodiments, the handle is not rotated axially when inserted into the nasal cavity of the subject. In some embodiments, the conduit of the navigation device comprises a material that is configured to allow the device to comply with the internal anatomy of the nasal cavity of the subject. A non-limiting example of a navigation device with a handle that is not required to be rotated during use is provided in FIG. 12.

In some embodiments, the lumen and channel of the conduit extends through the handle. For example, in some embodiments, the handle adjoins the conduit at the distal end of the conduit. In some embodiments, the handle comprises an opening at the distal end of the handle (e.g., or at the distal end of the body).

In some embodiments, the length of the handle of the conduit ranges from 10 mm to 135 mm. In some embodiments, the length of the handle of the conduit ranges from 10 mm to 40 mm. In some embodiments, the length of the handle of the conduit ranges from 10 mm to 45 mm. In some embodiments, the length of the handle is provided as “L3” of FIG. 1B. In some embodiments, the length of the handle is about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm. mm. In some embodiments, the handle of the conduit comprises a length of about 20 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, about 95 mm, about 100 mm, about 105 mm, about 110 mm, about 115 mm, about 120 mm, about 125 mm, about 130 mm, about 135 mm.

### 5.1.2. Depth Limiter

An aspect of the navigation device of the present disclosure includes a depth limiter (e.g., safety stopper).

In some embodiments, the depth limiter is non-adjustable and is inherent in the device and abuts or represents the distal end of the conduit or the abutting end of the handle. In some embodiments, the depth limiter is adjustable. In some embodiments, the depth limiter prevents the conduit from further extending within the nasal cavity once the depth limiter is pushed up against/adjacent to the nasal apex or surrounding anatomy of the nasal cavity.

In some embodiments, the depth limiter is positioned along a portion of the outer length of the conduit to limit insertion within the nasal cavity (12). As shown in FIG. 2, FIG. 11, and FIG. 12, depth limiter (12), when the navigation device is inserted into the nasal cavity, once touching the nasal apex or surrounding anatomy of the subject, the depth limiter prevents movement of the conduit. In some embodiments, the depth limiter is configured to restrict movement when first making contact with the nasal apex or surrounding anatomy. In some embodiments, the depth limiter is configured to restrict movement when compressed against the nasal apex until stopping against proximal cartilage structures.

In some embodiments, the depth limiter restricts the insertion depth of the conduit. In some embodiments, the depth limiter is configured to set the insertion depth based on the method of external anatomical measurement and extrapolation to the internal path length from the nasal apex to the olfactory cleft as described herein. In some embodiments, the depth limiter is positioned along the conduit based on an external measurement of the nasal anatomy with a conversion factor to determine depth limiter position. In some embodiments, the depth limiter is positioned on the conduit at the insertion depth position along the conduit. In some embodiments, the insertion depth ranges from 20 mm to 90 mm when secured in place. In some embodiments, the insertion depth ranges from 20 mm to 90 mm. In some embodiments, the insertion depth ranges from 10 mm to 100 mm. In some embodiments, the insertion depth ranges from 30 mm to 70 mm. In some embodiments, the insertion depth ranges from 40 mm to 80 mm. In some embodiments, the insertion depth ranges from 15 mm to 80 mm. In some embodiments, the insertion depth ranges from 40 mm to 90 mm when secured in place. In some embodiments, the insertion depth is about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, or about 90 mm

In some embodiments, the depth limiter is slidable when in an unlocked position along the outer length of the conduit. In some embodiments, the depth limiter is securable when in a locked position on the outer length of the conduit. In some embodiments, the locked position limits movement of the conduit until the insertion depth of the conduit is reached within the nasal cavity of the subject. In some embodiments, the depth limiter comprises a locking mechanism 13 configured to secure the depth limiter in place on the conduit once adjusted, thereby preventing or restricting movement or loosening of the depth limiter. In some embodiments, the depth limiter comprises a stepped locking mechanism, such that the depth limiter can be placed in a particular number of preset positions, ranging from 3 to 15 different positions.

In some embodiments, the distal end of the conduit is downstream of the depth limiter. In some embodiments, the proximal end of the conduit is upstream of the depth limiter, wherein the depth limiter separates the proximal region and the distal region of the conduit.

In some embodiments, the depth limiter is retained based on frictional contact between the interface of the conduit and depth limiter.

In some embodiments, the depth limiter is positioned along the conduit based on an external measurement of the nasal anatomy with a conversion factor to determine depth limiter position.

### 5.1.3. Insertion Device

An aspect of the navigation device includes an insertion device for inserting into the body of the navigation device that guides the insertion device into the olfactory cleft of the subject.

In some embodiments, the insertion device is a surgical tool. In some embodiments, the insertion device is a biopsy device. In some embodiments, the insertion device is a delivery tool configured to deliver a therapeutic agent to the olfactory region of the nasal cavity. In some embodiments, the navigation device is used to deliver a mist, liquid, or solid therapeutic agent with no need for an insertion device.

In some embodiments, the insertion device is a collection tool for collecting a sample from the olfactory cleft.

In some embodiments, the insertion device is configured to move through the lumen and/or channel of the conduit and into a particular region of the nasal cavity, such as the olfactory region of the nasal cavity of the subject. In some embodiments, the insertion device is a collection tool configured to collect the sample within the olfactory region of the nasal cavity.

In some embodiments, the insertion device is a commercially available brush. In some embodiments, the commercially available brush is selected from: ConMed® Cytology Brush 1.70 mm×160.00 cm, Olympus BC-201C-1006, Cook Medical BCB-5-120-2-S, Hobbs Medical, Inc. 4290 and 4291, Copan FLOQSwabs® Flocked Swabs 503CS01, and Puritan HydraFlock 25-3306-H.

### 5.1.3.1 Collection Tool-Collection Tip

In some embodiments, the collection tool includes a collection tip at the proximal end of the collection tool configured to collect the sample. In some embodiments, the collection tool further comprises a shaft extending from the collection tip.

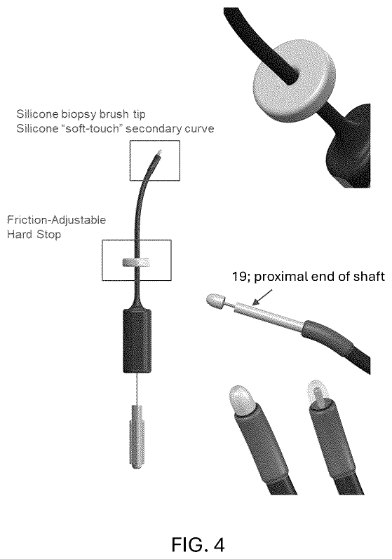

In some embodiments, the collection tool further comprises a handle attached to the distal end of the shaft. A non-limiting example of the collection tool is provided in FIG. 2 and FIG. 4. As shown in FIGS. 2 and 4, the collection tool comprises a shaft (19) (proximal end of shaft), (21) (distal end of shaft), a handle (18), and a collection tip (14). In some embodiments, the collection tip is configured to remove, detach, scrape, and/or collect the olfactory epithelium and/or nasal cells within the olfactory region of the nasal cavity.

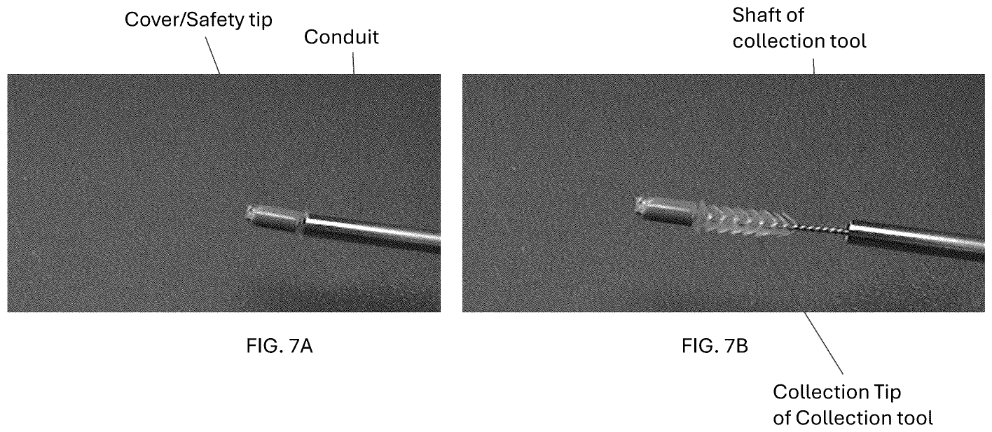

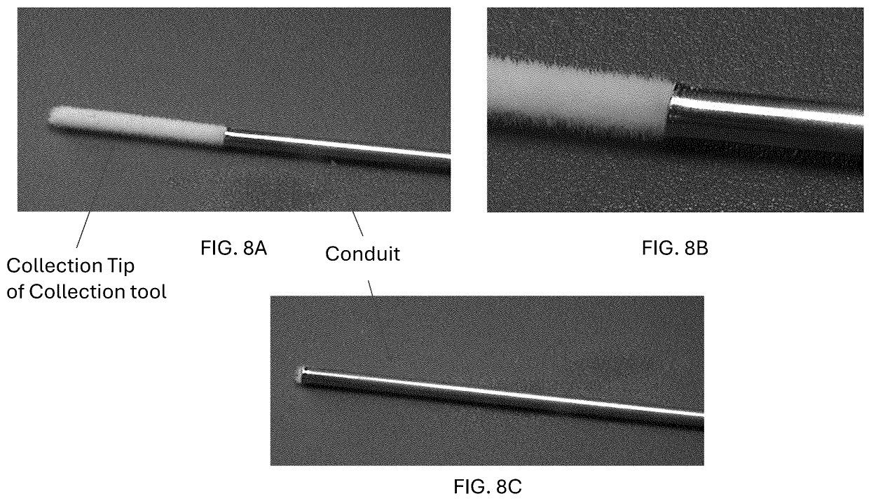

In some embodiments, the collection tip comprises a swab or brush at the proximal end of the collection tool. A non-limiting example of the swab or brush is provided in FIG. 7B and FIG. 8A. In some embodiments, the collection tip comprises bristles at the proximal end of the collection tool (e.g., FIG. 7B). In some embodiments, the collection tip comprises kerfs at the proximal end of the collection tool. In some embodiments, the collection tip comprises a debrider at the proximal end of the collection tool (e.g., FIG. 4). Non-limiting examples of the collection tool are provided in FIGS. 2, 7, and 8. FIG. 7B illustrates the collection tool in an extended position, e.g., where the collection tip and a portion of the shaft extend out of the lumen of the conduit. FIG. 7A illustrates a “retracted” or non-extended position where the collection tool is covered e.g., by a safety tip (FIG. 7A) or fully within the lumen of the conduit (FIG. 8C).

In some embodiments, the collection tool may include brush bristles, swab tips, felt, debriding blades, needles, adhesive, or a combination thereof, that incorporate specific geometry and material composition so as to optimize their collection of the desired cell types. In certain embodiments, the collection tool comprises a tip that may be a cylindrical metal or plastic (or similar material) tip with a hollow center and gratings on the side so that, when rotated, the gratings exfoliate the sides of the nasal cavity and collect the tissue in the hollow center. In some embodiments, these gratings may be shaped in such a way as to minimize damage when moved into and out the nasal cavity, only exfoliating tissue when rotated.

In some embodiments, the collection tool comprises a debrider comprising a cylindrical tube with cutouts forming teeth that work to remove tissue and draw it into the inner cavity of the device as it is rotated. In some embodiments, the debrider can remove, detach, or scrape the tissue and draw it into the inner cavity of the device as it is rotated.

In some embodiments, an adhesive, mastic, glue or the like can be added to the collection tool to adhere to the desired tissue and secure it to the sampling tip for removal from the body.

In some embodiments, collection tool includes a swab. In some embodiments, the swab of the navigation device may have kerfs that allow it to only bend in one plane, so that it is inserted vertically into the nostril and then only bends in the sagittal plane, so that it uses the roof of the path to guide it to the collection area. In some embodiments, this may be combined with bends in the axial plane that guide it against the septum or towards the right area.

In some embodiments, the collection tool (14) is contained within the lumen and/or channel of the conduit until the conduit is correctly placed within the olfactory cleft of the nasal cavity and once the depth limiter contacts the major alar cartilage and/or nasal apex of the nasal cavity. After the conduit tip is correctly placed within the olfactory cleft of the nasal cavity, the collection tool is deployed or extended out of the proximal end of the conduit (e.g., via proximal opening) and into the olfactory region of the nasal cavity.

In some embodiments, the collection tip of the collection tool comprises a length ranging from 5 mm to 20 mm. In some embodiments, the collection tip of the collection tool comprises a length ranging from 5 mm to 20 mm. In some embodiments, the collection tip of the collection tool comprises a length ranging from 1 mm to 5 mm. In some embodiments, the portion of the collection tool entering the olfactory region comprises a length ranging from 5 to 40 mm.

In some embodiments, the shaft of the collection tool comprises a length ranging from 50 to 250 mm. In some embodiments, the shaft of the collection tool comprises a length ranging from 50 to 120 mm. In some embodiments, the collection tip comprises a length ranging from 5 to 40 mm.

In some embodiments, the distal end of the shaft comprises a length ranging from 5 to 50 mm. In some embodiments, the distal end of the shaft comprises a length ranging from 0 to 50 mm. In some embodiments, the distal end of the shaft comprises a length ranging from 0 to 20 mm.

In some embodiments, the shaft of collection device is made from a material selected from one or more of: Polypropylene (PP), Polyethylene (PE), Polyurethane (PU), Nylon (Polyamide, PA), Polyether block amide (PEBA, Pebax), Polyester (PET, PETG), Flexible PVC (plasticized), Thermoplastic Elastomers (TPE, TPR), Thermoplastic Polyurethane (TPU), Silicone elastomer, Polystyrene (PS), Acrylonitrile Butadiene Styrene (ABS), Acrylic (PMMA), Stainless steel (medical-grade, e.g., 304 or 316), and Polysulfone (PSU, PPSU).

FIG. 11 provides an illustration of the steps using the navigation device according to one embodiment of the methods provided herein. Before the conduit is inserted into the nasal cavity, the length from the external nasion to the nasal apex of the subject is measured and a path length from the nasal apex to the olfactory cleft is determined as described herein to determine the insertion depth. The depth limiter is then adjusted and positioned on the outer length of the conduit based on the derived insertion depth. Step 1: The device is aligned coaxial with the nasal cavity and with “1” normal to the septal plane. The proximal end of the conduit is then inserted or advanced axially into the nasal cavity and toward the nasion of the subject until the user inserting the conduit feels a slight resistance (e.g., resistance of the conduit in contact with a wall within the nasal cavity) (steps 1-2). Once a slight resistance is felt by the user, the handle of the conduit is twisted or rotated from “1” to “2” (e.g., about 90 degrees) (step 3). Once the indicatory channel is normal to the septal plane and the number “2” on the handle is visible, the conduit is then further advanced or inserted until the depth limiter is in contact with the nasal apex of the subject (step 4). The insertion device is then extended or inserted within the lumen of the conduit until the handle of the insertion device is adjacent or touching the distal end of the handle of the conduit (Step 5; collection tool is advanced until fully extended) such that the insertion tip reaches the olfactory region of the subject's nasal cavity. In this non-limiting example, the insertion device is a collection tool. The collection tool is rotated or twisted a number of times to collect the sample (Step 6). The navigation device is then removed or retracted from the nasal cavity to the starting position (step 7). The device is then fully retracted with axial rotation allowed (Step 8).

In another embodiment, as shown in FIG. 12, the rotation of the navigation tool once it enters the nasal cavity of the subject is not required. FIG. 12 provides an illustration of the steps using the navigation device where the user does not rotate the handle of the navigation device to provide alignment into the olfactory region of the nasal cavity. Before the conduit is inserted into the nasal cavity, the length from the external nasion to the nasal apex of the subject is measured and a path length from the nasal apex to the olfactory cleft is determined as described herein to determine the insertion depth. The depth limiter is then adjusted and positioned on the outer length of the conduit based on the derived insertion depth. Step 1: The device is aligned coaxial with the nasal cavity. The proximal end of the conduit is then inserted or advanced axially into the nasal cavity until the depth limiter is in contact with the nasal apex of the subject (step 2). The insertion device is then extended or inserted within the lumen of the conduit until the handle of the insertion device is adjacent or touching the distal end of the handle of the conduit (Step 3; collection tool is advanced until fully extended) such that the insertion tip reaches the olfactory region of the subject's nasal cavity. In this non-limiting example, the insertion device is a collection tool. The collection tool is rotated or twisted a number of times to collect the sample (Step 4). The navigation device is then removed or retracted from the nasal cavity to starting position (step 5). The device is then fully retracted with axial rotation allowed (Step 6).

As described herein, the collection tip of the collection tool is configured to collect samples from the olfactory region of the nasal cavity. In some embodiments, the sample comprises olfactory epithelium. In some embodiments, the sample comprises nasal cells. In some embodiments, the nasal cells are olfactory cells. In some embodiments, the olfactory cells are selected from: olfactory sensory neurons, olfactory supporting cells, olfactory ensheathing cells (OECs), olfactory epithelial cells, mesenchymal stem cells, and basal (stem) cells.

In some embodiments, the olfactory region is the olfactory cleft of the nasal cavity. In some embodiments, the olfactory region comprises olfactory filaments, the olfactory epithelium, and/or the olfactory mucosa.

### 5.1.3.2 Collection Tool-Shaft

Aspects of the collection tool comprise a shaft connecting to or extending from the distal end of the collection tip. In some embodiments, the length of the shaft 50 to 250 mm. In some embodiments, the length of the shaft 50 to 120 mm. In some embodiments, the length of the shaft ranges from 75 to 250 mm. In some embodiments, the length of the shaft ranges from 100 to 250 mm. In some embodiments, the length of the shaft ranges from 125 to 250 mm. In some embodiments, the length of the shaft ranges from 150 to 250 mm. In some embodiments, the length of the shaft ranges from 175 to 250 mm. In some embodiments, the length of the shaft ranges from 200 to 250 mm. In some embodiments, the length of the shaft ranges from 225 to 250 mm. e

In some embodiments, the distal end of the shaft remains in the lumen during sample collection. In some embodiments, the distal end of the shaft has a length ranging from 5 to 40 mm. In some embodiments, the distal end of the shaft has a length ranging from 5 to 30 mm. In some embodiments, when not in use, the collection tip and the proximal end of the shaft are within the lumen of the conduit, and the distal end of the shaft protrudes distally out of the distal end of the lumen (e.g., distal opening of the conduit handle).

In some embodiments, the shaft region of the sample collection tool can be made of any known material. In some embodiments, the shaft region of the sample collection is made from one or more materials selected from: Polypropylene (PP), Polyethylene (PE), Polyurethane (PU), Nylon (Polyamide, PA), Polyether block amide (PEBA, Pebax), Polyester (PET, PETG), Flexible PVC (plasticized), Thermoplastic Elastomers (TPE, TPR), Thermoplastic Polyurethane (TPU), Silicone elastomer, Polystyrene (PS), Acrylonitrile Butadiene Styrene (ABS), Acrylic (PMMA), Stainless steel (medical-grade, e.g., 304 or 316), and Polysulfone (PSU, PPSU).

### 5.1.3.3 Collection Tool-Handle

In some embodiments, the collection tool comprises a handle connected to or attached to the distal end of the shaft.

In some embodiments, the handle is configured to rotate the collection tool between 90 and 360 degrees to collect the sample. In some embodiments, the handle is rotated 360 degrees at least 1 time, at least 2 times, at least 3 times, at least 4 times, at least 5 times, at least 6 times, at least 7 times, at least 8 times, at least 9 times, or at least 10 times.

### 5.1.4. Other Features of the Navigation Device

In some embodiments, the navigation device can include a navigation aid that includes a magnetic (hall, etc.) sensor that is attached externally, to the bridge of the nose, or somewhere on the head, to facilitate navigation of the sampling device. In some embodiments, the collection tool may include a magnetic material or otherwise emit a magnetic field detectable by the sensor. This would allow the user to know, or be guided to, the correct position within the anatomy for the sample collection.

In some embodiments, the sampling device can emit a magnetic field while utilizing a magnet placed somewhere else (not on the intranasal portion of the device) and utilizing a ferrous material for a portion or all of the handle. In one embodiment, a magnet is placed in the base of the handle well outside the sampling area and the magnetic effect is carried into the sampling area by the ferrous nature of the handle material.

In some embodiments, the navigation aid may include an audio, visual, audiovisual, or tactile alert when the internal portion of the device reaches a certain depth(s), when it leaves a proper path or area, or when a certain amount of force is exerted through the device.

In some embodiments, a navigation aid is included in the sample collection tip that uses the distance to the septum to track a path towards the collection area. For example, as the tip stays along the septum and goes the correct distance, the tip self-navigates to the correct collection location. In some embodiments, movement of the tip into the correct collection location may be done with sensors, such as a pressure sensor to maintain contact, or a spatial sensor such as a LIDAR sensor to show distance from the septum wall.

In some embodiments, the navigation device may include a sheath or cover that covers the navigation device when not in use, before and/or after use. In some embodiments, the sheath may be retracted during use to facilitate the deployment of the navigation device to the target anatomy.

In some embodiments, the sheath may contain, or be made from, an indicating material, or material containing an indicating material, or incorporate a window to allow viewing of an indicating material, to allow the user to positively confirm the sample was properly collected.

In some embodiments, the sheath may be bent, as to navigate the nasal cavity and direct the deployed device towards the collection area. In some embodiments, the conduit of the navigation device is a sheath.

In some embodiments, the navigation device may have an asymmetric or otherwise featured handle to facilitate rotation of the handle, and therefore the sampling tip.

In some embodiments, the navigation device may have a depth stop, adjustable or fixed, that limits the depth to which the sampling device may be extended, and/or retracted. This depth stop(s) may be at the proximal, distal, or both ends of the device.

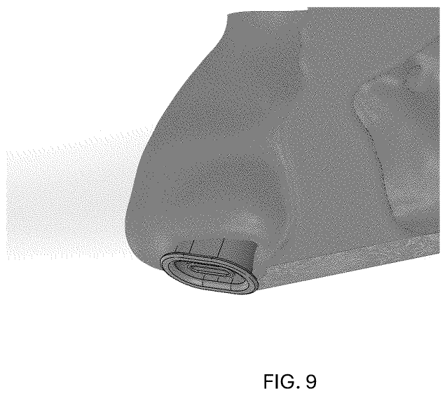

In some embodiments, navigation device may include a guiding device to help guide the navigation device into nasal cavity. A non-limiting example of a guiding device is provided in FIG. 9. In some embodiments, the guiding device is the conduit of the navigation device.

In some embodiments, the guiding device (e.g., conduit of the navigation device) protects sampled mucosa and protects the sampling device (e.g., collection tool) from inadvertently sampling the wrong area. In some embodiments, the guiding device is a soft material that may be customized into different sizes and geometries for different nostril and nasal anatomies. In some embodiments, the conduit and/or guiding device comprises a designed guided lumen in the center. In some embodiments, the conduit and/or guiding device comprising the guide may have a lip that sits on the outside of the nostril controlling its depth of placement and/or direction and/or distance from the sides of the nostril. In some embodiments, the conduit and/or guiding device comprising the guide may have geometric features that make it highly flexible and supple, while still providing adequate rigidity and guidance for the sampling device.

In some embodiments, the navigation device is coated with a biocompatible lubricant to smooth insertion of the navigation device and protect the nasal cavity. In some embodiments, this is a water-based, sterile lubricant (e.g., Surgilube, lubricating jelly, gel, surgical lubricant). In some embodiments, the navigation device is sealed in a water-tight sterile package with the lubricant pre-applied. In some embodiments, the lubricant is added prior to use by the user.

In some embodiments, the safety tip is a bead, blunt tip, or the like that is on the tip of the brush, swab, or other sampling device that helps to guide the sampling device along the anatomy of the nasal cavity and to stop the navigation device from penetrating too deeply into tissue if the device is mis-used.

In some embodiments, the navigation device may include a radio-opaque, contrast, MR fiducial, or other construction to allow imaging or other location of the navigation device within the nasal cavity.

5.2. Methods for Using a Navigation Device to Insert an Insertion Device into the Nasal Cavity of a Subject

Aspects of the present disclosure include methods for navigating an insertion device within the nasal cavity of a subject.

Aspects of the present disclosure include methods for collecting a sample within the olfactory cleft of a subject using a navigation tool and an insertion device.

An aspect of the present disclosure includes a method of inserting an insertion device in the nasal cavity of a subject, the method includes (1) inserting a conduit of a navigation device of the present disclosure into the nasal opening of the subject; (2) once the conduit is in the olfactory cleft of the nasal cavity, inserting or passing an insertion device through the lumen of the conduit and into the olfactory region of the nasal cavity of the subject.

An aspect of the present disclosure includes a method of collecting a sample in the nasal cavity of a subject, the method includes (1) inserting a conduit of a navigation device of the present disclosure into the nasal opening of the subject; (2) once the conduit is in the olfactory cleft of the nasal cavity, inserting or passing an insertion device comprising a collection tool through the lumen of the conduit and into the olfactory region of the nasal cavity of the subject; and (3) collecting the sample by rotating the collection tool.

An aspect of the present disclosure includes a method of collecting a sample in the nasal cavity of a subject, the method includes (1) inserting a conduit of a navigation device of the present disclosure into the nasal opening of the subject; (2) after the conduit tip is past the nasion of the nasal cavity, inserting or passing an insertion device comprising a collection tool through the lumen of the conduit and into the olfactory region of the nasal cavity of the subject; and (3) collecting the sample by rotating the collection tool.

In some embodiments, the method comprises repeating step (3) at least 1 time, at least 2 times, at least 3 times, at least 4 times, or at least 5 times.

In some embodiments, the collection tool is rotated about 90 degrees. In some embodiments, the collection tool is rotated about 100 degrees. In some embodiments, the collection tool is rotated about 120 degrees. In some embodiments, the collection tool is rotated about 180 degrees. In some embodiments, the collection tool is rotated about 200 degrees. In some embodiments, the collection tool is rotated about 240 degrees. In some embodiments, the collection tool is rotated about 300 degrees. In some embodiments, the collection tool is rotated about 360 degrees. In some embodiments, the collection tool is rotated about 90-360 degrees at least once, at least twice, at least 3 times, at least 4 times, or at least 5 times.

In some embodiments, the method comprises rotating the conduit with a handle along its longitudinal axis.

In some embodiments, rotating the conduit with the handle occurs once the proximal end of the conduit of the navigation device hits a wall within the nasal cavity. In some embodiments, rotating the conduit with the handle occurs when there is resistance against the navigation device. In some embodiments, the method does not comprise rotating the conduit with a handle along its longitudinal axis.

In some embodiments, the proximal end of the collection tool is fully extended through the lumen and channel when the handle of the collection tool is adjacent to or touching the distal end of the handle of the conduit. In some embodiments, the collection tool is fully extended through the lumen and channel when the handle of the collection tool is adjacent to the distal end of the handle of the conduit. By “fully extended”, the method comprises fully extending the proximal region of the collection tool that is necessary to collect the sample from the olfactory region.

In some embodiments, the method comprises removing the navigation device from the nasal cavity of the subject. In certain embodiments, removing the navigation device comprises retracting the collection tool distally away from the olfactory region of the subject's nasal cavity and fully within the lumen and channel of the conduit; and removing the conduit of the navigation device out of the nasal cavity of the subject.

In some embodiments, removing the conduit of the navigation device out of the nasal cavity of the subject comprises rotating the conduit

In some embodiments, the method comprises placing the collection tool in a buffer.

In some embodiments, the sample comprises olfactory tissue. In some embodiments, the sample comprises nasal cells. In some embodiments, the nasal cells are olfactory cells. In some embodiments, the olfactory cells are selected from: olfactory receptor cells, olfactory supporting cells, olfactory ensheathing cells (OECs), and basal (stem) cells. In some embodiments, the olfactory region is the olfactory cleft of the nasal cavity. In some embodiments, the olfactory region comprises olfactory filaments, and/or the olfactory mucosa.

In some embodiments, the method further comprises analyzing the sample. In some embodiments, analyzing the sample comprises determining the viability of the olfactory cells within the sample.

In some embodiments, the method comprises, before (1) inserting a proximal end of a conduit of a navigation device of the present disclosure into the nasal opening of the subject, measuring the length of the external nasion to the nasal apex of the subject to determine the insertion depth of the navigation device.

In some embodiments, the method comprises using a subsample or portion of the eluted material or entire sample to qualify the collection, such as to determine the presence or absence of certain olfactory tissue or olfactory cells.

In some embodiments, the insertion depth is the length at which the depth limiter is positioned on the outer position of the conduit.

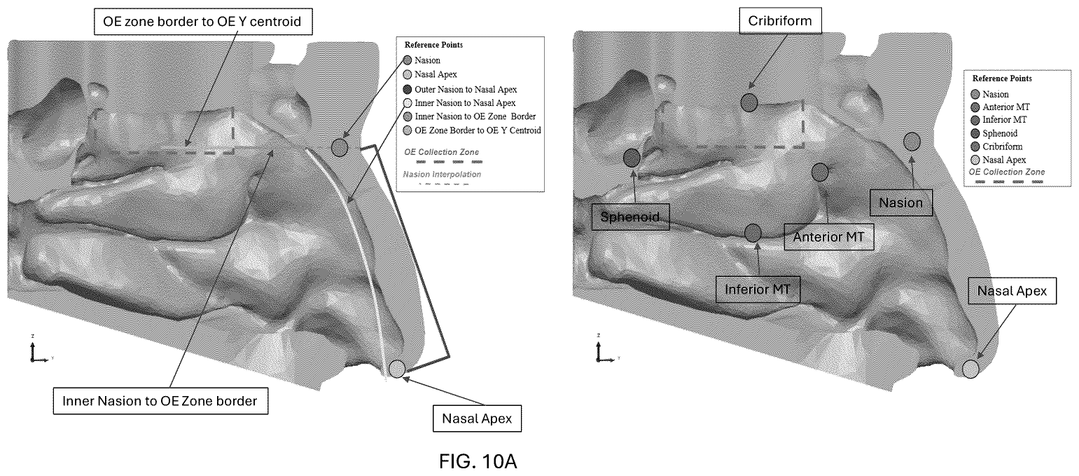

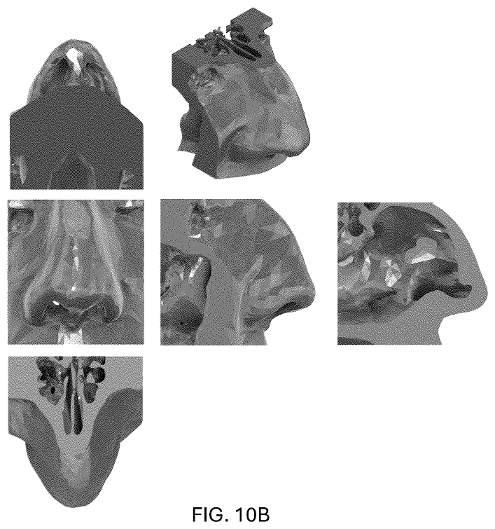

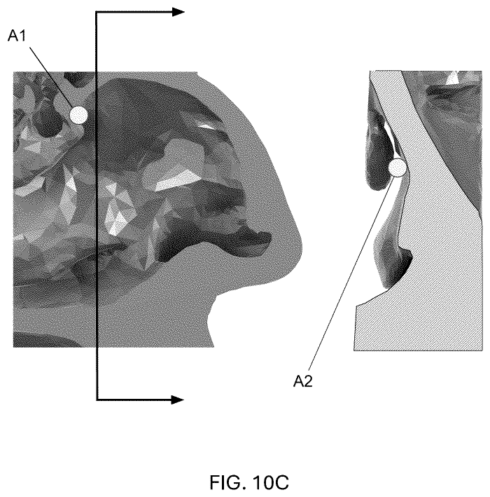

In some embodiments, the navigation device does not enter the anterior middle turbinate attachment point “A1” and the middle meatus “A2” regions of the nasal cavity as shown in FIG. 10C. In some embodiments, the navigation device is configured to prevent the insertion device from entering the anterior middle turbinate attachment point “A1” of the nasal cavity as shown in FIG. 10C. In some embodiments, the navigation device is configured to prevent the insertion device from entering the middle meatus “A2” of the nasal cavity as shown in FIG. 10C. In some embodiments, curves C1, C2, C3 and angles A1 and A2 as shown in FIG. 1B of the navigation device prevent entering the middle meatus and the anterior middle turbinate attachment point of the nasal cavity by shifting the tip of the navigation device toward the septum.

A non-limiting example of the method of using the navigation device to collect a sample is provided by FIG. 11.

FIG. 11 provides an illustration of the steps using the navigation device according to one embodiment of the methods provided herein. Before the conduit is inserted into the nasal cavity, the length from the external nasion to the nasal apex of the subject is measured and a path length from the nasal apex to the olfactory cleft is determined as described herein to determine the insertion depth. The depth limiter is then adjusted and positioned on the outer length of the conduit based on the derived insertion depth, or a specific device length with a fixed depth limiter is appropriately selected. Step 1: The device is aligned coaxially with the nasal cavity and with “1” normal to the septal plane. The proximal end of the conduit is then inserted or advanced axially into the nasal cavity and toward the nasion of the subject until the user inserting the conduit feels a slight resistance (e.g., resistance of the conduit in contact with a wall within the nasal cavity) (steps 1-2). Once a slight resistance is felt by the user, the handle of the conduit is twisted or rotated from “1” to “2” (e.g., about 90 degrees) (step 3). Once the indicatory channel is normal to the septal plane and the number “2” on the handle is visible, the conduit is then further advanced or inserted until the depth limiter is in contact with the nasal apex of the subject (step 4). The insertion device is then extended or inserted within the lumen of the conduit until the handle of the insertion device is adjacent or touching the distal end of the handle of the conduit (Step 5; collection tool is advanced until fully extended) such that the insertion tip reaches the olfactory region of the subject's nasal cavity. In this non-limiting example, the insertion device is a collection tool. The collection tool is rotated or twisted a number of times to collect the sample (Step 6). The navigation device is then removed or retracted from the nasal cavity to starting position (step 7). The device is then fully retracted with axial rotation allowed (Step 8).

Another non-limiting example of the method of using the navigation device to collect a sample is provided by FIG. 12.

FIG. 12 provides an illustration of the steps using the navigation device according to one embodiment of the methods provided herein. Before the conduit is inserted into the nasal cavity, the length from the external nasion to the nasal apex of the subject is measured and a path length from the nasal apex to the olfactory cleft is determined as described herein to determine the insertion depth. The depth limiter is then adjusted and positioned on the outer length of the conduit based on the derived insertion depth, or a specific device length with a fixed depth limiter is appropriately selected. Step 1: The device is aligned coaxially with the nasal cavity. The proximal end of the conduit is then inserted or advanced axially into the nasal cavity until the depth limiter is in contact with the nasal apex of the subject (step 2), signifying the tip of the conduit has reached the olfactory cleft. The insertion device is then extended or inserted within the lumen of the conduit until the handle of the insertion device is adjacent or touching the distal end of the handle of the conduit (Step 3; collection tool is advanced until fully extended) such that the insertion tip is fully extended within the olfactory cleft. In this non-limiting example, the insertion device is a collection tool. The collection tool is rotated or twisted a number of times to collect the sample (Step 4). The navigation device is then removed or retracted from the nasal cavity to starting position (step 5). The device is then fully retracted with axial rotation allowed (Step 6).

Aspects of the present disclosure include methods for collecting and/or detecting olfactory epithelium tissue and/or olfactory cells in a sample using an accessory tool to the device as described herein. In some embodiments, the accessory tool determines an amount of OE issue and/or olfactory cells in absolute terms or relative to other tissues. In some embodiments, the device determines the presence of OE tissue and/or olfactory cells above a threshold, and communicates that result quickly (e.g., within 40 minutes) to the test practitioner. In some embodiments, detection of OE tissue and/or olfactory cells is determined by direct or amplified detection of RNAs, proteins, or metabolites that are specific to or in differentiable levels in OE tissue and/or olfactory cells (OCs), including combinations of these factors. In some embodiments, detection can be determined by Olfactory Marker Protein/gene, or other proteins and genes found specifically in neurons, olfactory neurons, neural progenitor cells, or other OCs. These factors may be detected through any method. In some embodiments, the readout or output results of the test can include, but is not limited to: colorimetric/color change, lateral flow, electronic, or one of a variety of other assay methods that allows the user to ascertain the result. In some embodiments, based on this result, the practitioner may resample the subject or subject while they remain at the clinical care setting.

In some embodiments, a portion (e.g., an amount) of the sample collected is used for detection/quantification of OE tissue and/or OCs within the sample. In some embodiments, an amount of the sample may be taken from the rest of the sample using any known method, such as taking a portion of the liquid in which a sample is distributed, using a portion of the navigation device, or taking a subsample of the sample. In other embodiments, no specific amount of the sample is used, and the accessory tool is inherent to the navigation device, which itself determines whether collection of the sample was successful.

In some embodiments, a user can use the accessory tool to determine whether a sample collection was successful and may repeat testing (e.g., once or multiple times) if the result indicates that collection did not successfully collect any or enough OE tissue and/or OCs. In some embodiments, the accessory tool can also detect whether the OE tissue and/or OCs collected is viable for use in culture, sequencing, or other conventional analysis techniques. In some embodiments, the accessory tool and methods of the present disclosure allow for the repeat collection to be done immediately in the case of an insufficient sample, while the subject or participant remains at the point of care. In some embodiments, the device and methods of the present disclosure allow for repeat collection that increases the consistency and reliability of OE tissue and/or OC sample collection. In some embodiments, the accessory tool of the present disclosure can be used in combination with any other navigation devices for OE/OC samples.

In some embodiments, the sample navigation device, accessories, and methods described herein may be used in outsubject specialist or non-specialist care, such as, but not limited to: otorhinolaryngologists (ENTs), general practitioners, psychiatrists, and other non-ENTs.

In some embodiments, the method is to confirm the presence of OE tissue or olfactory cells above a quantity threshold, and communicate that result quickly (e.g., within 40 minutes) to the user. In some embodiments, the user can use the analysis to determine whether a sample collection was successful and may repeat testing (e.g., once or multiple times) if the result indicates that collection did not successfully collect any or enough OE tissue or olfactory cells.

### 5.3. Systems

An aspect of the present disclosure comprises a system for navigating an insertion device into the olfactory cleft of a subject. In some embodiments, the system includes the navigation device described in the present disclosure configured to insert into the olfactory cleft, and the insertion device described in the present disclosure configured to insert into the conduit of the navigation device to reach the olfactory cleft, and one or more additional components.

In some embodiments, the one or more additional components comprise a nasal passage expander device. In some embodiments, the nasal expander device is a nasal strip. In some embodiments, the nasal strips can provide dilation at the nasal valve to ease the navigation device insertion and removal by increasing effective cross-section of the nasal cavity.

In some embodiments, the one or more additional components comprise nasal sprays, mists, or medications to be applied prior to use of the device. In some embodiments, these components comprise medications including oxymetazoline, tetracaine, lidocaine, combinations therein, or other decongestants, vasoconstrictors, anesthetics, and analgesics. The decongestants and vasoconstrictors may be used to clear nasal matrix from the navigated areas and constrict the nasal tissues to widen the paths, and the anesthetics and analgesics reduce potential pain for the subject.

In some embodiments, the one or more additional components is a nasal spray. In some embodiments, the nasal spray is a vasoconstrictor. In some embodiments, the vasoconstrictor is oxymetazoline. In some embodiments, the nasal spray is an anesthetic. In some embodiments, the anesthetic is tetracaine or lidocaine.

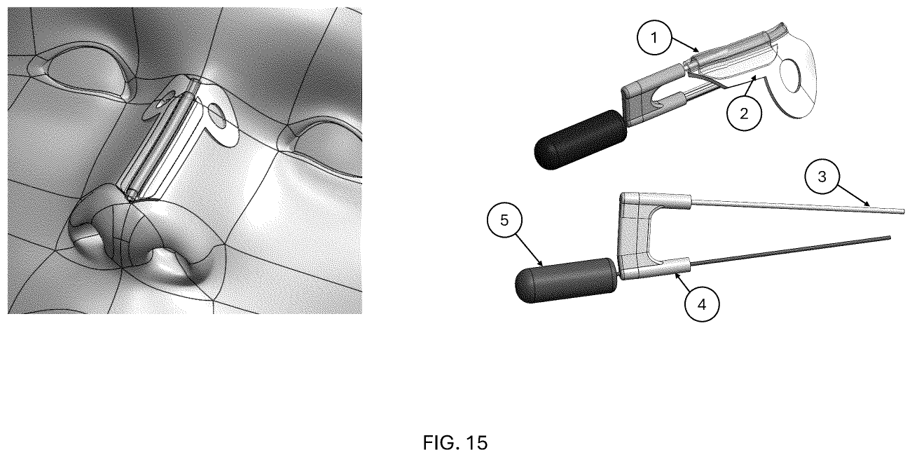

In some embodiments, one or more additional components comprise a self-adhesive guide device. In some embodiments, the one or more additional components is a nasal dilator. In some embodiments, the self-adhesive device guides provide additional support and guidance to the existing navigation device. A non-limiting example includes a self-adhesive tape similar to 3M Tegiderm temporarily adhered to the subject's nose during collection. Non-limiting examples are shown in FIG. 15. As shown in FIG. 15, this accessory comprises a tube with one or more lumens (1), a patch of self-adhesive tape (2), and a guide rod (3) with a lumen at a preset angle (4). The adhesive (2) is adhered to the subject's nasal ridge. The guide rod (3) is inserted into (2) until resting against the nasal apex. The insertion device, such as a biopsy device is inserted through (4) and kept at a set angle relative to the nasal bridge as a result.

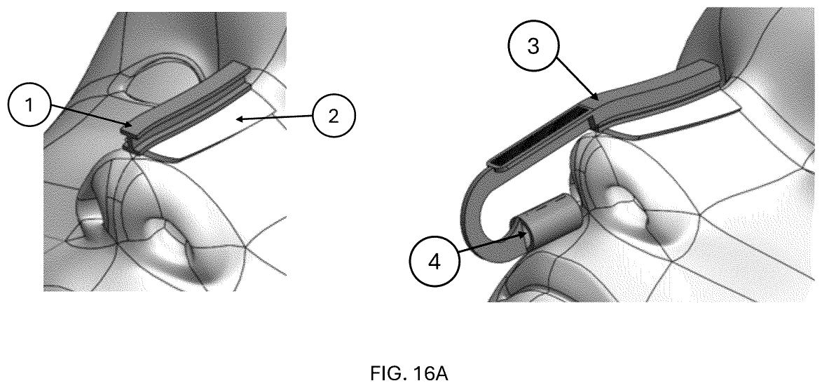

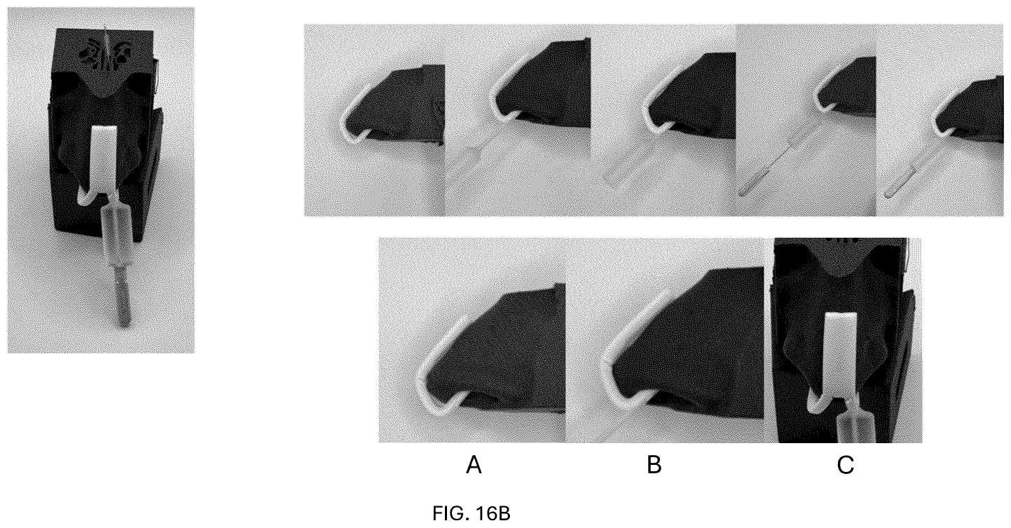

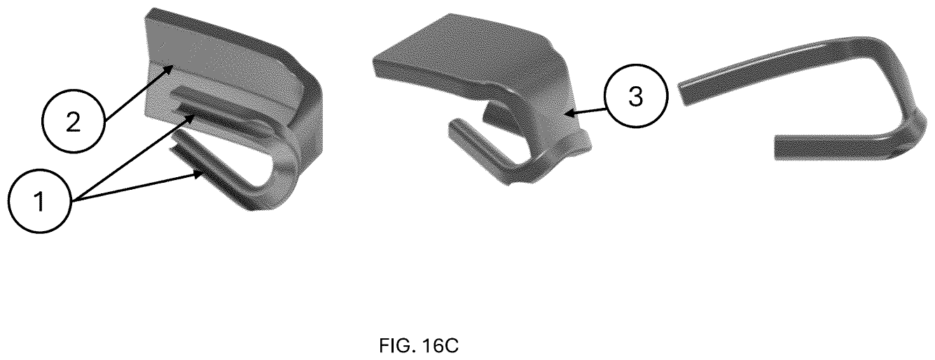

In some embodiments, the navigation device is configured to be used with the self-adhesive device guide as shown in FIGS. 16A-16C. The accessory component as shown in FIG. 16A includes a flexible beam (1), a self-adhesive patch (2), and a guide sheath (3) containing a lumen oriented at a preset angle (4) to accommodate the navigation device and insertion device. The adhesive patch (2) is applied to the subject's nasal ridge. The guide sheath (3) is advanced along the flexible beam (1) until it rests against the nasal apex. The insertion device such as a biopsy device is then inserted through the angled lumen (4), ensuring it remains at a consistent, predefined angle relative to the nasal bridge.

The accessory component is a self-adhesive guiding device as shown in FIGS. 16B-16C comprises two channels (1) with undercut semicircular cross-sections, oriented at a predetermined angle, and a self-adhesive patch (2). Each arm (1) is inserted into a nostril until the distal end (3) contacts the nasal apex, at which point the adhesive patch (2) is secured to the subject's nasal ridge. An insertion device such as a biopsy device is then inserted through one or both angled channels (1), maintaining a consistent, predefined angle relative to the nasal bridge throughout the procedure. FIG. 16A, (A) shows the accessory advanced until in contact with the nasal apex, while (B) and (C) show the biopsy device advanced at a predefined angle relative to the nasal bridge.

### 5.4. Kits

Aspects of the present disclosure include a kit carrying out the methods described herein.

An aspect of the present disclosure includes a kit comprising a navigation device of the present disclosure.

In some embodiments, the kit further comprises an insertion tool of the present disclosure.

In some embodiments, the kit further comprises one or more additional components to facilitate use or guidance of the navigation device.

In some embodiments, one or more additional components is selected from: a self-adhesive guide device, nasal dilator, the accessory tool for determining successful collection, nasal sprays, and the like.

In some embodiments, the self-adhesive guide device is a self-adhesive guide device of any one of the self-adhesive guide devices shown in FIGS. 15-16.

In some embodiments, the kit also includes one or more additional components which are buffers (media) and receptacles to store and process the sample after collection.

In some embodiments, the kit comprises one or more additional components configured to aid int eh analysis steps of the methods described herein.

### 6. EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1: Function and Testing of Deflecting Tip

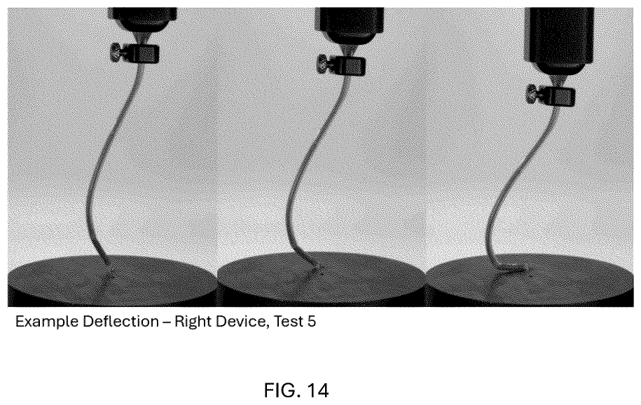

To simulate and calculate the pressure applied by the navigation device to the nasal cavity during insertion of the navigation device into the nasal cavity, the navigation device was placed in a compressive load with a fix-pin buckling condition as shown in FIG. 14. The navigation device is compressed 15 mm until the deflectable tip collapses on the plate (“Example Deflection”-right image). Two navigation devices were tested-one Left version, one Right version. Each device was compressed 10 times.

Pressure values were calculated from the cross-sectional area of the tip.

Result: The force applied, and pressure values calculated based on the cross-sectional area of the tip were within safety limits, thus determining that the device prevents damage to the cribriform plate and sphenoid of the nasal cavity.

### 7. Equivalents and Incorporation by Reference

While the invention has been particularly shown and described with reference to a preferred embodiment and various alternate embodiments, it will be understood by persons skilled in the relevant art that various changes in form and details can be made therein without departing from the spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

All references, issued patents and patent applications cited within the body of the instant specification are hereby incorporated by reference in their entirety, for all purposes.

1. A navigation device comprising:
a body comprising:
a conduit comprising a proximal end, a distal end, and a lumen extending therethrough, wherein the proximal end of the conduit comprises a tip configured to be inserted into a nasal opening of a subject;
a handle adjoining to the distal end of the conduit and configured to: insert the proximal end of the conduit into the olfactory cleft of the subject.

2. The navigation device of claim 1, wherein a depth limiter positioned along a portion of the outer length of the conduit restricts further movement of the conduit through the nasal cavity of the subject when the depth limiter is in contact with the nasal tip, apex, or major alar cartilage of the subject.

3. (canceled)

4. The navigation device of claim 1, wherein the navigation device is made from one or more biocompatible polymer materials configured to:
provide one or more regions of stiffness along the length or cross-section of the conduit, and
prevent the navigation device from exerting an amount of pressure on the nasal cavity or cribriform plate of the subject;
wherein the one or more biocompatible polymer materials is selected from:
Polyetheretherketone (PEEK), Polycarbonate (PC), Polyetherimide (PEI, Ultem), Polysulfone (PSU), Polyphenylsulfone (PPSU), Polyamide (Nylon, PA), Acrylic (PMMA), Polyethylene Terephthalate (PET), Rigid Polyvinyl Chloride (Rigid PVC), Acrylonitrile Butadiene Styrene (ABS); or
Ethylene-vinyl acetate (EVA), Polyolefin (PO), Polyether block amide (PEBA, Pebax), Thermoplastic elastomers (TPE), Thermoplastic polyurethane (TPU), Silicone elastomer (Silicone), and Flexible polyvinyl chloride (Flexible PVC).

5. (canceled)

6. The navigation device of claim 4, wherein the device's external conduit surface comprises a low surface energy material to reduce friction during device insertion, and wherein the amount of pressure is 750 kPa to 4500 kPa, and wherein the device's internal conduit surface comprises a low surface energy material to reduce friction against the insertion device during use.

7. (canceled)

8. (canceled)

9. (canceled)

10. (canceled)

11. (canceled)

12. The navigation device of claim 4, wherein the navigation device remains usable when a force applied to the navigation device is less than 10 newtons, less than 9 newtons, less than 8 newtons, less than 7 newtons, less than 6 newtons, less than 5 newtons, less than 4 newtons, or less than 3 newtons.

13. The navigation device of claim 1, wherein the body comprises a proximal opening at the proximal end of the conduit and a distal opening at the distal end of the handle, and the conduit comprises a first curved region at the proximal end of the conduit, wherein the first curved region comprises a radius of curvature ranging from 20 mm to 175 mm.

14. (canceled)

15. (canceled)

16. (canceled)

17. (canceled)

18. The navigation device of claim 1, wherein the proximal end of the conduit comprises a second curved region near the deflection tip, and wherein the second curved region comprises a radius of curvature ranging from 5 mm to 120 mm.

19. (canceled)

20. (canceled)

21. The navigation device of claim 18, wherein the proximal end of the conduit comprises a third curved region comprising a radius of curvature ranging from 20 mm to 150 mm.

22. (canceled)

23. The navigation device of claim 1, wherein the conduit comprises two or more parallel lumens, one central lumen configured to receive the insertion device and at least one adjacent lumen configured to receive a guidewire, and wherein tension applied to the guidewire selectively adjusts the curvature of the conduit in a predetermined region to facilitate alignment of the proximal end with the olfactory cleft.

24. (canceled)

25. The navigation device of claim 7, wherein the deflection tip comprises a flared tip.

26. (canceled)

27. The navigation device of claim 1, wherein the lumen of the conduit comprises a channel comprising a sample retention tip.

28. (canceled)

29. The navigation device of claim 1, wherein the outer diameter of the proximal opening of the conduit ranges from 0.25 mm to 6 mm.

30. The navigation device of claim 1, wherein the conduit is inserted within the nasal cavity until an insertion depth is reached, wherein the insertion depth is determined by a calculation based on the external nasal length of the subject's nose, wherein the external nasal length is measured by a sum of the length of the external nasion to the nasal apex of the subject.

31. (canceled)

32. (canceled)

33. (canceled)

34. (canceled)

35. The navigation device of claim 30, wherein the depth limiter is positioned at a location along the outer length of the conduit such that, when the conduit is advanced into the nasal cavity of the subject, the depth limiter restricts the insertion depth of the conduit ranging from 40 mm to 90 mm, and wherein the depth limiter is slidable when in an unlocked position and securable when in a locked position at a selected position on the outer region of the conduit to limit movement of the conduit until the insertion depth of the conduit is reached.

36. (canceled)

37. (canceled)

38. (canceled)

39. (canceled)

40. (canceled)

41. (canceled)

42. (canceled)

43. (canceled)

44. The navigation device of claim 1, wherein the handle is a barrel-containing space comprising a spring configured to limit the force of insertion of the conduit within the nasal cavity of the subject.

45. (canceled)

46. (canceled)

47. (canceled)

48. The navigation device of claim 1, wherein the lumen and channel of the conduit extends through the handle, wherein the handle adjoins the conduit at the distal end of the conduit, and wherein the handle comprises an opening at the distal end of the handle.

49. The navigation device of claim 1, wherein the navigation device further comprises an insertion device, wherein the insertion device is configured to move through the lumen of the conduit and into the olfactory region of the nasal cavity of the subject, wherein the insertion device is selected from:
a collection tool configured to collect nasal cells and/or olfactory tissue;
a delivery tool configured to deliver a therapeutic agent to the olfactory region of the nasal cavity;
a surgical tool; or
an imaging tool.

50. (canceled)

51. (canceled)

52. (canceled)

53. (canceled)

54. (canceled)

55. (canceled)

56. (canceled)

57. The navigation device of claim 49, wherein the collection tool comprises:
a collection tip at the proximal end of the collection tool configured to collect the sample comprising nasal cells and/or olfactory tissue;
a shaft extending from the collection tip; and
a handle attached to a distal end of the shaft.

58. The navigation device of claim 57, wherein the collection tip comprises:
a swab or brush at the proximal end of the collection tool;
a debrider at the proximal end of the collection tool; or
bristles at the proximal end of the collection tool.

59. (canceled)

60. (canceled)

61. (canceled)

62. (canceled)

63. (canceled)

64. The navigation device of claim 1, wherein the navigation device is configured as a single-use disposable device for sterility and to prevent cross-contamination.

65. The navigation device of claim 1, wherein the navigation device is coated with a biocompatible sterile lubricant for use.

66. (canceled)

67. The navigation device of claim 49, wherein the collection tool is within the lumen and a channel of the conduit, and wherein the collection tool is fully extended through the lumen and channel when the handle of the collection tool is adjacent to the distal end of the handle of the conduit.

68. (canceled)

69. A method of collecting a sample from the nasal cavity of a subject, the method comprising:
(1) inserting a proximal end of a conduit of a navigation device of claim 1 into the nasal opening of the subject, wherein one side of the conduit is adjacent to the septum of the nasal cavity;
(2) after the conduit tip is past the nasion of the nasal cavity, inserting the insertion device comprising a collection tool through the lumen of the conduit and into the olfactory region of the nasal cavity of the subject; and
(3) collecting the sample by rotating the collection tool.

70. The method of claim 69, wherein the collection tool is rotated between 0.5 to 20 complete rotations.

71. The method of claim 69, wherein the method further comprises, before step (2), rotating the conduit with the handle between 5-90 degrees along its longitudinal axis, wherein the proximal end of the conduit is inserted into the nasion of the nasal cavity of the subject until the depth limiter restricts further movement of the conduit through the nasal cavity.

72. (canceled)

73. (canceled)

74. The method of claim 69, wherein the method comprises removing the navigation device from the nasal cavity of the subject by retracting the collection tool distally away from the olfactory region of the subject's nasal cavity and fully within the lumen and channel of the conduit; and rotating the conduit of the navigation device to remove the navigation device out of the nasal cavity of the subject.

75. (canceled)

76. (canceled)

77. (canceled)

78. The method of claim 69, wherein the sample comprises olfactory tissue and/or nasal cells selected from one or more of: olfactory sensory neurons, olfactory supporting cells, olfactory ensheathing cells (OECs), olfactory epithelial cells, mesenchymal stem cells, and basal (stem) cells.

79. (canceled)

80. (canceled)

81. (canceled)

82. (canceled)

83. (canceled)

84. (canceled)

85. (canceled)

86. (canceled)

87. The method of claim 69, wherein the method comprises using a subsample or portion of the sample to qualify the collection, such as to determine the presence or absence of certain olfactory tissue or olfactory cells, and wherein the method further comprises: determining the presence of olfactory epithelial tissue or olfactory cells above a quantity threshold within 40 minutes.

88. (canceled)

89. (canceled)

90. (canceled)

91. The method of claim 69, wherein the method comprises, before (1) inserting a proximal end of a conduit of a navigation device of claim 1 into the nasal opening of the subject, measuring the length of the external nasion to the nasal apex of the subject to determine the insertion depth of the navigation device, wherein the insertion depth is the length at which the depth limiter is positioned on the outer position of the conduit.

92. (canceled)

93. A method of navigating an insertion device through a nasal cavity of a subject, the method comprising:
(1) inserting a proximal end of a conduit of a navigation device of claim 1 into the nasal opening of the subject, wherein one side of the conduit is adjacent to the septum of the nasal cavity;
(2) once the conduit is in the nasion of the nasal cavity, inserting the insertion device through the lumen of the conduit and into the olfactory region of the nasal cavity of the subject; and
(3) performing a function intended by the insertion device.

94. (canceled)

95. (canceled)

96. (canceled)

97. (canceled)

98. A system for navigating a navigation device into the nasal cavity of a subject, the system comprising:
a navigation device of claim 1;
an insertion device configured to be placed through the lumen of the conduit and into the olfactory region of the nasal cavity of the subject to perform its intended function;
one or more additional components configured to aid the navigation device to be inserted into the nasal cavity of the subject.

99. The system of claim 98, wherein the one or more additional components is selected from:
a nasal valve dilator,
a self-adhesive guide device, and
a nasal spray comprising oxymetazoline, tetracaine, or lidocaine.

100. (canceled)

101. (canceled)

102. (canceled)

103. A kit for navigating a navigation device into the nasal cavity of a subject, the kit comprising:
a navigation device of claim 1.

104. (canceled)

105. (canceled)

106. (canceled)

107. (canceled)

108. (canceled)

109. (canceled)
